SEQUENCE LISTING (11 sequences)

SEQ ID NO: 1 - DNA, 23 bp, Artificial Sequence
  Generic CRE FP
gcggtctggcagtaaaaactatc

SEQ ID NO: 2 - DNA, 23 bp, Artificial Sequence
  Generic CRE RP
gtgaaacagcattgctgtcactt

SEQ ID NO: 3 - DNA, 20 bp, Artificial Sequence
  KEAP1flox FP
cgaggaagcgtttgctttac

SEQ ID NO: 4 - DNA, 20 bp, Artificial Sequence
  KEAP1flox RP
gagtcaccgtaagcctggtc

SEQ ID NO: 5 - DNA, 19 bp, Artificial Sequence
  KEAP1 deletion FP
gagtccacagtgtgtggcc

SEQ ID NO: 6 - DNA, 20 bp, Artificial Sequence
  KEAP1 deletion RP
gagtcaccgtaagcctggtc

SEQ ID NO: 7 - DNA, 2726 bp, Homo sapiens
gggatgtgcccgggctgcgggcatcgcgggccgcgagcgcccctgcggccgtgcccggagaccaggaaaacgggcgccacggcccagggcgcctccgagttccccgccaggactcggagggccaggagggcgcgacctgggtggatatttttgttggacggcgcaactcttggggtggcccgggagcggcggaaaccgagcgagagaaccaggaggcgctgcgcagaaggaggcccgggggctccgaggcgttgaggggctcgatctgcgttctggggtccctgagtgccagaggtggtggtgttgcttatcttctggaaccccatgcagccagatcccaggcctagcggggctggggcctgctgccgattcctgcccctgcagtcacagtgccctgagggggcaggggacgcggtgatgtacgcctccactgagtgcaaggcggaggtgacgccctcccagcatggcaaccgcaccttcagctacaccctggaggatcataccaagcaggcctttggcatcatgaacgagctgcggctcagccagcagctgtgtgacgtcacactgcaggtcaagtaccaggatgcaccggccgcccagttcatggcccacaaggtggtgctggcctcatccagccctgtcttcaaggccatgttcaccaacgggctgcgggagcagggcatggaggtggtgtccattgagggtatccaccccaaggtcatggagcgcctcattgaattcgcctacacggcctccatctccatgggcgagaagtgtgtcctccacgtcatgaacggtgctgtcatgtaccagatcgacagcgttgtccgtgcctgcagtgacttcctggtgcagcagctggaccccagcaatgccatcggcatcgccaacttcgctgagcagattggctgtgtggagttgcaccagcgtgcccgggagtacatctacatgcattttggggaggtggccaagcaagaggagttcttcaacctgtcccactgccaactggtgaccctcatcagccgggacgacctgaacgtgcgctgcgagtccgaggtcttccacgcctgcatcaactgggtcaagtacgactgcgaacagcgacggttctacgtccaggcgctgctgcgggccgtgcgctgccactcgttgacgccgaacttcctgcagatgcagctgcagaagtgcgagatcctgcagtccgactcccgctgcaaggactacctggtcaagatcttcgaggagctcaccctgcacaagcccacgcaggtgatgccctgccgggcgcccaaggtgggccgcctgatctacaccgcgggcggctacttccgacagtcgctcagctacctggaggcttacaaccccagtgacggcacctggctccggttggcggacctgcaggtgccgcggagcggcctggccggctgcgtggtgggcgggctgttgtacgccgtgggcggcaggaacaactcgcccgacggcaacaccgactccagcgccctggactgttacaaccccatgaccaatcagtggtcgccctgcgcccccatgagcgtgccccgtaaccgcatcggggtgggggtcatcgatggccacatctatgccgtcggcggctcccacggctgcatccaccacaacagtgtggagaggtatgagccagagcgggatgagtggcacttggtggccccaatgctgacacgaaggatcggggtgggcgtggctgtcctcaatcgtctcctttatgccgtggggggctttgacgggacaaaccgccttaattcagctgagtgttactacccagagaggaacgagtggcgaatgatcacagcaatgaacaccatccgaagcggggcaggcgtctgcgtcctgcacaactgtatctatgctgctgggggctatgatggtcaggaccagctgaacagcgtggagcgctacgatgtggaaacagagacgtggactttcgtagcccccatgaagcaccggcgaagtgccctggggatcactgtccaccaggggagaatctacgtccttggaggctatgatggtcacacgttcctggacagtgtggagtgttacgacccagatacagacacctggagcgaggtgacccgaatgacatcgggccggagtggggtgggcgtggctgtcaccatggagccctgccggaagcagattgaccagcagaactgtacctgttgaggcacttttgtttcttgggcaaaaatacagtccaatggggagtatcattgtttttgtacaaaaaccgggactaaaagaaaagacagcactgcaaataacccatcttccgggaagggaggccaggatgcctcagtgttaaaatgacatctcaaaagaagtccaaagcgggaatcatgtgcccctcagcggagccccgggagtgtccaagacagcctggctgggaaagggggtgtggaaagagcaggcttccaggagagaggcccccaaaccctctggccgggtaataggcctgggtcccactcacccatgccggcagctgtcaccatgtgatttattcttggatacctgggagggggccaatgggggcctcagggggaggccccctctggaaatgtggttcccagggatgggcctgtacatagaagccaccggatggcacttccccaccggatggacagttattttgttgataagtaaccctgtaattttccaaggaaaataaagaacagactaactagtgtctttca

SEQ ID NO: 8 - protein, 624 aa, Homo sapiens
MQPDPRPSGAGACCRFLPLQSQCPEGAGDAVMYASTECKAEVTPSQHGNRTFSYTLEDHTKQAFGIMNELRLSQQLCDVTLQVKYQDAPAAQFMAHKVVLASSSPVFKAMFTNGLREQGMEVVSIEGIHPKVMERLIEFAYTASISMGEKCVLHVMNGAVMYQIDSVVRACSDFLVQQLDPSNAIGIANFAEQIGCVELHQRAREYIYMHFGEVAKQEEFFNLSHCQLVTLISRDDLNVRCESEVFHACINWVKYDCEQRRFYVQALLRAVRCHSLTPNFLQMQLQKCEILQSDSRCKDYLVKIFEELTLHKPTQVMPCRAPKVGRLIYTAGGYFRQSLSYLEAYNPSDGTWLRLADLQVPRSGLAGCVVGGLLYAVGGRNNSPDGNTDSSALDCYNPMTNQWSPCAPMSVPRNRIGVGVIDGHIYAVGGSHGCIHHNSVERYEPERDEWHLVAPMLTRRIGVGVAVLNRLLYAVGGFDGTNRLNSAECYYPERNEWRMITAMNTIRSGAGVCVLHNCIYAAGGYDGQDQLNSVERYDVETETWTFVAPMKHRRSALGITVHQGRIYVLGGYDGHTFLDSVECYDPDTDTWSEVTRMTSGRSGVGVAVTMEPCRKQIDQQNCTC

SEQ ID NO: 9 - DNA, 4469 bp, Homo sapiens
agacccacgccctgctccctccgcccggcacctgcaggaagggctggaactgcctctgcgtacccgccgcccgtttccgccctcccgctcctcccacgcgtgccgcccgggaccccgcagcaccgctgccccgatccgagccctccacccccactccggtccccctcctctcttcccggaagcgcggcgcgtggcggcccggcggcgcggattggacgcgtggcacctacagagacacccgggggggtgggacggaggtgagcgagcgcccgcggaggatgcggtggggagccagctccgggagctgcccgcggtcgcgcgtggggccgtgcacgcggtggggggaagcgcgtgcccttctccaagcgcgcaccccgccgccgagcccgtgagccctcgtagggtggtggccgcggcgagtagaggtaggggtcgcccgcggccggcgccccgggactcttattgtgacagggtggcgcgctgtgcttagtcaccgtgacccgcgcggcggaggcggaggcagagcgcggccatggcggggtgagtgagccgctccaggccgcggcccgggaccaggccctgcgggctctcccggcgtcagggctgcgcctccgagcggtggggaggccgctggagcaggcgccgggtaccgggcggccgctgcacagccccctgcgcaatgccaggcccgagctccggcagtgtggtcacgcgtgacagtcgctcactagctggggcccctggagcatttcatcccccccctccccacggtgatctaatagacaaaacacgcggagtcgcgactccaggctgagcccagaacctggggagccagacgcagaccctctcttgtctccccacatcttctttgaaagcataattcctcccctggccccaggtctccaagggtctcctgaatccctccccgtgggtgttccagatgctgcacactctcttgccccaggagcttggtgttcgcttagtgtttcctatacagaccttgctttatttttaggccttttctgtcttctcgctgtgtctctggagctcagagcagtcccaaatacataaatggcaggctctgtaaatgttggtgtggtgttgaaaggaatctgacatgttggacgaaggcaaggggagggaaggatggctggaacagtgaagaggttggaaagcgggtgtggagttttacaggccattgacgatttggggtttccattcttgggctcggtgaaaggtgttgggtgattctgagcaggaaaaggaacatgatatgccctgaaggcccgcgagttgagaagttagtttgaatggagccggctgtgtccagtttacttggcttggcaaaatctgcacttagatatcattgcttagtcttgcaaaaaaagaagcctggctggacatggtggcacatactgtaatcccagcactcgggaggagccagctttggttgcatagtgagttggaagccagcctaggctatgtaagaccctgtctcaaataaaataaaataaagtggcagggtctggtctaacccagcctctgttcccagcgctgtgctcttccctccctccaggcccctaacggctagcagaggaactgtgtcttgtcatcaggaaccccatgcagcccgaacccaagcttagcggggctccccgcagcagccagttcctgcccctgtggtcaaagtgccccgagggggccggggacgcagtgatgtatgcctccacggagtgcaaggcagaggtgacgccctcgcaggacggtaaccgaaccttcagctacacactagaggatcacaccaagcaggcttttggcgtcatgaacgagcttcgcctgagccagcaactctgtgacgtgaccctgcaggtcaaatatgaggacatcccagctgcccaattcatggctcacaaagtggtgctggcctcctccagcccagtctttaaagccatgttcaccaacgggcttcgggagcagggcatggaggtggtgtccatcgaaggcatccaccctaaggtcatggaaaggcttattgagttcgcctacacggcctccatctccgtgggcgagaagtgtgtcctgcacgtgatgaacggggcggtcatgtaccagattgacagcgtggttcgagcctgcagcgacttcctcgtgcagcagctggaccccagcaacgccattggcatcgccaacttcgcggagcagatcggctgcactgaactgcaccagcgtgcccgggagtatatctacatgcacttcggggaggtggccaagcaggaggagttcttcaacctgtcacactgccagctggccacgctcatcagccgggatgatctgaacgtacgctgcgagtccgaggtgttccacgcgtgcatcgactgggtcaaatacgactgcccgcagcggcgcttctacgtgcaggcactgctgcgggccgtgcgctgccatgcgctcacgccgcgcttcctgcagacgcagctgcagaagtgtgagatcctgcaggccgacgcgcgctgcaaggactacctggtgcagatattccaggagctcacgctgcacaagcccacgcaggcagtgccctgccgcgcgcccaaagtgggccgcctcatctacacagcgggcggttacttccgacagtcgctcagctacctggaggcctacaacccgagcaatggctcctggctgcgcctggccgatctacaggtgccgcgcagtgggctggcaggctgcgtggtgggtgggctgctatacgctgtgggcggccgcaacaactctccggatggcaacactgactccagcgccctggactgctacaaccccatgaccaaccagtggtcgccctgtgcctctatgagcgtgccacgcaaccgcatcggggtgggggtcatagatggccacatctacgcagtcgggggttcccacggctgcatccaccacagcagcgtggagagatatgagccagagcgggacgagtggcatctagtcgcgccaatgttgacacggaggattggcgtgggcgtggcagtgctcaaccgcttgctgtatgcagtggggggctttgacgggactaaccggcttaactccgcagaatgttactatccagagaggaatgagtggcggatgatcacaccgatgaataccatccggagcggggccggggtctgcgtgctgcacaactgtatctatgcagcagggggctacgatgggcaggaccagttgaacagtgtggagcgctacgacgtggagacagagacctggactttcgtagcccccatgaggcatcaccgtagtgcgctggggattactgtgcaccagggcaagatctacgtcctcggaggctatgatggccacacttttctggacagtgtggaatgctatgacccggacagtgatacctggagtgaggtgacccgcatgacatctggccgcagcggggtgggtgtggccgtcaccatggaaccctgtcggaagcaaattgatcaacaaaactgtacctgctgaagcacttggaatacctgagcactgacaacaggacagaaaaacagtctgtgtatcactgcttctctgtactaaagaaaaaagaagaaaacaaagcataaacagaaaacacagggccgaagaggcggcagaagaagtcatcccttcttccaggaagggcgactgggatgccttgtaaaggaccttgtggaagaccagaactcaaatccatgggcccatctgtcatagccctggagcgtccaagtctgggatggggtatgggcggggcaccctcacaggtgagaagcccttgaactcccaccaccagaaggggggggacaggcaaagcaggagatcacatgtttttttctttggttcctgcaactcggtgatcaattccagtggacaggggaagaagggacagctgaggccaaggggctgaggctccctctggaactggggcccaagggacaagccggcacagagaagcctctgggctctgagccctgaacagttattttgttaaataaccctgtaagtttcccatgggaataaagaatggagtaggcacacaggtcttcagagggcggtcggaatccctcagggagagacagctcttctattgaaatacacgcagatcctgatggggctggtatctgaaacccgtctattgtctctgcttgccattgtacattctgctcagacagggcatcttgcttcttgtgggacacacagttgtctgtcagtttcagggcattagaagccaatgacctaacttctgtgcctcctaacttctcctggggcctcctgtgtttagctttattttgaggcagggactcacgtcgtccgggatggccttcagttcagaccttgaactgacgctgccgcctgtcccagcctaccgagtgctggggctacatctgtatagcgcaatgcctggttcctgcttattatttttgtacccaagcaggaaaataaaggtttctgggacattg

SEQ ID NO: 10 - DNA, 686 bp, Homo sapiens
gtggccaagcaggaggagttcttcaacctgtcacactgccagctggccacgctcatcagccgggatgatctgaacgtacgctgcgagtccgaggtgttccacgcgtgcatcgactgggtcaaatacgactgcccgcagcggcgcttctacgtgcaggcactgctgcgggccgtgcgctgccatgcgctcacgccgcgcttcctgcagacgcagctgcagaagtgtgagatcctgcaggccgacgcgcgctgcaaggactacctggtgcagatattccaggagctcacgctgcacaagcccacgcaggcagtgccctgccgcgcgcccaaagtgggccgcctcatctacacagcgggcggttacttccgacagtcgctcagctacctggaggcctacaacccgagcaatggctcctggctgcgcctggccgatctacaggtgccgcgcagtgggctggcaggctgcgtggtgggtgggctgctatacgctgtgggcggccgcaacaactctccggatggcaacactgactccagcgccctggactgctacaaccccatgaccaaccagtggtcgccctgtgcctctatgagcgtgccacgcaaccgcatcggggtgggggtcatagatggccacatctacgcagtcgggggttcccacggctgcatccaccacagcagcgtggagag

SEQ ID NO: 11 - DNA, 206 bp, Homo sapiens
atatgagccagagcgggacgagtggcatctagtcgcgccaatgttgacacggaggattggcgtgggcgtggcagtgctcaaccgcttgctgtatgcagtggggggctttgacgggactaaccggcttaactccgcagaatgttactatccagagaggaatgagtggcggatgatcacaccgatgaataccatccggagcggggccg

(19) United States
(12) Patent Application Publication
(10) Pub. No.: US 2016/0120158 A1
(43) Pub. Date: May 5, 2016

(54) COMPOSITIONS AND METHODS FOR THE STUDY AND TREATMENT OF ACUTE KIDNEY INJURY

(71) Applicant: The Johns Hopkins University, Baltimore, MD (US)

(72) Inventors: Hamid Rabb, Ellicott City, MD (US); Sanjeev Noel, Baltimore, MD (US); Maria N. Martina-Lingua, Baltimore, MD (US); Lorraine C. Racusen, Marriottsville, MD (US); Abdel Rahim Hamad, Ellicott City, MD (US); Samatha Bandapalle, Sterling, VA (US)

(21) Appl. No.: 14/930,883

(22) Filed: Nov. 3, 2015

Related U.S. Application Data
(60) Provisional application No. 62/074,255, filed on Nov. 3, 2014.
(60) Provisional application No. 62/074,825, filed on Nov. 4, 2014.

(51) Int. Cl. A01K 67/027; A61K 35/17

(52) U.S. Cl. CPC A01K 67/0276; A61K 35/17; A01K 2217/075; A01K 2227/10; A01K 2227/105; A01K 2267/035

(57) ABSTRACT

The present invention relates to the field of nephrology. More specifically, the present invention provides compositions and methods useful for the study and treatment of acute kidney injury. In one embodiment, the present invention provides a knockout animal whose genome comprises a deletion of exon 2 and exon 3 of kelch-like ECH-associated protein 1 (KEAP1) in T-cells. In another embodiment, a method for treating a subject diagnosed with AKI comprising the steps of (a) isolating T-cells from the subject; (b) activating Nrf2 expression in the isolated T-cells; and (c) administering the T-cells back to the subject.

### CROSS-REFERENCE TO RELATED APPLICATIONS

This application claims the benefit of U.S. Provisional Application No. 62/074,825, filed Nov. 4, 2014, and U.S. Provisional Application No. 62/074,255, filed Nov. 3, 2014, each of which is incorporated herein by reference in its entirety.

### STATEMENT OF GOVERNMENTAL INTEREST

This invention was made with government support under grant no. DK084445, awarded by the National Institutes of Health. The government has certain rights in the invention.

### FIELD OF THE INVENTION

The present invention relates to the field of nephrology. More specifically, the present invention provides compositions and methods useful for the study and treatment of acute kidney injury.

### INCORPORATION-BY-REFERENCE OF MATERIAL SUBMITTED ELECTRONICALLY

This application contains a sequence listing. It has been submitted electronically via EFS-Web as an ASCII text file entitled “P13210-03_ST25.txt.” The sequence listing is 17,781 bytes in size, and was created on Oct. 29, 2015. It is hereby incorporated by reference in its entirety.

### BACKGROUND OF THE INVENTION

Ischemia reperfusion (IR) induced acute kidney injury (AKI) is associated with significant mortality in native kidneys and worsens outcomes after kidney transplantation. Excessive oxidative stress, apoptosis, epithelial and endothelial cell dysfunction, and inflammation are among the important pathophysiological mechanism during ischemic AKI. There is a great need for research tools and therapeutic approaches to study and treat AKI.

### SUMMARY OF THE INVENTION

CD4+ T lymphocytes play a major pathophysiological role in ischemia reperfusion and drug-induced acute kidney injury. Currently there are no treatment options available to treat acute kidney injury. Recent research work in our lab has demonstrated protective role of Nrf2 during ischemia-reperfusion and Cisplatin-induced acute kidney injury. Nrf2 is an important transcription factor that regulates the expression of multiple antioxidant and xenobiotic genes. We further demonstrated that pharmacological activation of Nrf2 regulated antioxidant response using small molecule activators such as CDDO-Im can protect from acute kidney injury. However administration of an Nrf2 activator such as CDDO-Me results in systemic activation of anti-oxidant response that may have deleterious side effects especially during prolonged administration.

We therefore generated mice with CD4+ T cell specific activation of Nrf2 in order to explore the possibility of T cell specific Nrf2 activation on acute kidney injury. We found significant protection from ischemia-reperfusion induced acute kidney injury in these mice. This finding has immense clinical implication in protecting and treating ischemia related organ injuries during transplantation, myocardial infraction, stroke, hemorrhage, cardiac arrest and many other oxidative stress and inflammation driven diseases. Nrf2 in T cells can be activated using ex-vivo pharmacologic and genetic approaches and reintroduced into subjects before and/or after the induction of ischemia-reperfusion injury to further explore T-cell based therapy in prevention and treatment of acute kidney injury. In further embodiments, activated human T cells either from patients or matched, healthy volunteers using Nrf2 activator(s) are administered during or after an ischemic event. This approach provides a more natural and tolerable treatment strategy to protect organs from ischemia-reperfusion injury.

Accordingly, in one aspect, the present invention provides knockout animals. In one embodiment, the present invention provides a knockout animal whose genome comprises a deletion of exon 2 and exon 3 of kelch-like ECH-associated protein 1 (KEAP1). In particular embodiments, the genome comprises a deletion of exon 2 and exon 3 of KEAP1 in T-cells of the animal. The sequence for KEAP1 is publicly available, NCBI Reference Sequence: NM_016679.4, GeneID:50868. In a specific embodiment, KEAP1 is encoded by the nucleic acid sequence of SEQ ID NO:7. In another embodiment, KEAP1 is encoded by the nucleic acid sequence of SEQ ID NO:9. In yet another embodiment, exon 2 is encoded by the nucleic acid sequence of SEQ ID NO:10. In a further embodiment, exon 3 is encoded by the nucleic acid sequence of SEQ ID NO:11. In another embodiment, the animal exhibits lower or no expression of KEAP1 as compared to a wildtype animal. In a specific embodiment, the animal is a mouse. In an alternative embodiment, the animal is a rat. The present invention also provides a population of T-cells derived or isolated from a knockout animal described herein.

In another aspect, the present invention provides methods for treating subject. In one embodiment, a method comprises the steps of (a) activating Nrf2 in T-cells isolated from a subject; and (b) administering the T-cells of step (a) to the subject. In a specific embodiment, the subject is a human. In particular embodiment, the subject suffers from acute kidney injury (AKI). In another embodiment, the subject suffers from ischemia reperfusion induced AKI. The present invention also provides a method for treating a subject diagnosed with AKI comprising the steps of (a) isolating T-cells from the subject; (b) activating Nrf2 expression in the isolated T-cells; and (c) administering the T-cells back to the subject. In certain embodiments, the AKI comprises ischemia reperfusion induced AKI. In particular embodiments, the activation step is accomplished by contacting the T-cells with an Nrf2 activator. Nrf2 activators are described herein and include, but are not limited to, sulforaphane, tert-butylhydroquinone (tBHQ), Protandim, Cddo-Im, CDDO-Me, Oltipraz (4-methyl-5-(2-pyrazinyl)-3-dithiolethione), bardoxolone methyl, dihydro-CDDO-trifluoroethyl amide (dh404), resveratrol, chalcone, a chalcone derivative, anethole dithiolethione, 6-methylsulphinylhexyl isothiocyanate, curcumin, caffeic acid phenethyl ester, and 4′-bromoflavone. In other embodiments, T-cells can be engineered to comprise a knock out of the Keap1 gene, for example, using the CRISPR/Cas9 technology.

In yet another embodiment, a method for treating a subject diagnosed with AKI comprises the step of administering to the subject autologous T-cells that were previously isolated from the subject and treated ex-vivo to activate Nrf2 expression. In other embodiments, a method for treating a patient having an ischemia-related injury comprises the steps of administering the subject autologous T-cells that were previously isolated from the subject and treated ex-vivo to activate/upregulate Nrf2 expression. In particular embodiments, the ischemia-related injury comprises organ injuries suffered during transplantation, myocardial infraction, hemorrhage, cardiac arrest and other oxidative stress and inflammation driven diseases. The ex-vivo treatment step can be accomplished by contacting the T-cells with an Nrf2 activator as described herein. The present invention can also applied in ischemia-reperfusion injury, stroke and drug induced tissue injury to other organs such as heart, lung, liver, brain, and spinal cord.

### BRIEF DESCRIPTION OF THE FIGURES

FIG. 1A-1F. Generation and characterization of CD4-Keap1-KOmice. (A) CD4-Cremice are crossed with Keap1F/F mice to generate CD4-Keap1-KO mice. (B) Mice are genotyped to confirm the presence of the Cre and Keap1 floxed allele using CRE and floxed primers. Lanes in B represent the following: lane 1, 100-bp DNA ladder; lane 2, 324-bp internal positive control showing CD4-Cre-negative mice; lane 3, 324-bp internal positive control and 100-bp Cre showing CD4-Cre-positive mice; lane 4, 383-bp Keap1 floxed allele in Keap1F/F mice; and lane 5, 383-bp Keap1 floxed allele in CD4-Keap1-KO mice. (C) CD4-Cre-mediated deletion of exons 2 and 3 of Keap1 is further confirmed by using deletion specific primers. Lanes in C represent the following: lane 1, 1-Kb DNA ladder; lane 2, 2954-bp WT Keap1 allele; and lane 3, 288-bp truncated Keap1 allele after deletion of exons 2 and 3. (D) Deletion of Keap1 significantly upregulates the expression of Nrf2 targets Nqo1 (P≦0.001), Ho-1 (P=0.05), and Gclc (P≦0.01) in T cells; however, there is no change in Nrf2 and Gclm mRNA levels. (E) Western blot analysis of Nrf2 and Nqo1 in nuclear and cytoplasmic fractions of T cells isolated from CD4-Keap1-KO (n=3) and Keap1F/F mice (n=3). (F) Quantification of Nrf2 and Nqo1 levels in nuclear and cytoplasmic fractions. Data represent the mean6SD. *P≦0.05; **P≦0.01; ***P≦0.001.

FIG. 2A-2E. Baseline characteristics of T cells in CD4-Keap1-KO mice. (A-C) T cell-specific augmentation of Nrf2 results in higher percentages of CD25+Foxp3+Tregs (A) and lower percentages of CD11b+CD11c+ and F4/80+ cells in CD4-Keap1-KO kidneys at baseline compared with Keap1F/F kidneys (B and C). (D) The percentage of CD69+CD4, CD8, and DNT cells is lower in kidneys of CD4-Keap1-KO mice than in Keap1F/F mice. (E) Percentages of CD4, CD8, and DNT cells for baseline intracellular TNF-a, IFN-g, and IL-17 are lower in kidneys of CD4-Keap1-KO mice compared with Keap1F/F mice. Representative flow images show selected populations and corresponding graphs show average percentages from four independent experiments. Data represent the mean6SD. *P≦0.05; **P≦0.01.KMNC, kidney mononuclear cell.

FIG. 3A-3C. Frequency of Tregs and intracellular cytokines by lymphocytes isolated from inguinal LN and thymus at baseline. (A) The percentage of Tregs is significantly higher in the LN in CD4-Keap1-KO at baseline than in Keap1F/F mice. (B and C) Baseline intracellular TNF-a, IFN-g, and IL-17 is lower in CD4, CD8, and DNT cells isolated from CD4-Keap1-KO LN (B) and thymus (C) than in Keap1F/F counterparts. Data represent the mean6SD. *P≦0.05; **P≦0.01.

FIG. 4A-4E. Effect of T cell-specific Keap1 deletion on IR-induced AKI. (A) Deletion of Keap1 from T cells in CD4-Keap1-KO mice (n=7) improves kidney function after bilateral IR injury compared with Keap1F/F mice (n=9). (B) There is no mortality in CD4-Keap1-KO mice; however, 20% of mice died in the control group 72 hours after IR injury. (C) Representative images of hematoxylin and eosin-stained kidney sections showing significantly fewer necrotic tubules and greater normal renal cortex and medullary tissue in CD4-Keap1-KO mice compared with Keap1F/F mice 24 and 72 hours after IR injury. (D) Dot plot showing the percent score for necrotic tubules and normal cortex and medulla for CD4-Keap1-KO (n=8-10) and Keap1F/F (n=9-11) mice 24 and 72 hours after IR injury. (E) Pro-inflammatory cytokine IFN-g is lower in whole kidney lysates of CD4-Keap1-KO mice compared with Keap1F/F mice 72 hours after IR injury, whereas TNF-a, MCP-1, and IL-10 are not significantly different between the groups. Graphs represent the mean6SEM. *P≦0.05; **P≦0.01. MCP-1, monocyte chemoattractant protein-1. Original magnification, 3200 in C.

FIG. 5A-5C. Post-IR changes in kidney-infiltrating immune cells and cytokine production in CD4-Keap1-KO and Keap1F/F mice. (A) There is a significantly higher percentage of Tregs (P=0.04) and a lower percentage of CD11b+CD11c+(P=0.02) and F4/80+(P=0.03) cells in kidneys of CD4-Keap1-KO mice 24 hours after the induction of AKI. (B) Absolute numbers of Tregs (343.306102.5 versus 284.1680.9) and CD11b+CD11c+(6.4310461.83103 versus 8.3310463.23103) and F4/80+(131056 4.13103 versus 1.8310569.13103) cells are not different between CD4-Keap1-KO and Keap1F/F mice at 24 hours after IR injury. (C) Intracellular IL-17 levels are higher in CD4, CD8, and DNT cells isolated 24 hours after IR from kidneys of CD4-Keap1-KO mice, whereas there is no difference in TNF-a and IFN-g production. IRI, ischemia reperfusion injury. Data represent the mean6SD. *P≦0.05

FIG. 6. In vitro activation of CD4+ T cells from spleens of CD4-Keap1-KO mice with anti-CD3/CD28 show attenuated IFN-g production at day 3 (P=0.03) and day 7 (P=0.05) compared with Keap1F/F. There is no difference in IL-4-producing CD4+ T cell populations in either mouse. Data represent the mean6SD. *P≦0.05.

FIG. 7A-7C. Effect of adoptive transfer of CD4-Keap1-KO T cells into WT (C57BL/6) mice (n=7-10). (A) The success of adoptive transfer is confirmed by establishing the presence of CFSE-labeled T cells in peripheral blood of WT recipients before inducing AKI. (B and C) Adoptive transfer of T cells from CD4-Keap1-KO mice significantly improves renal function (P=0.02) and improves survival (log-rank [Mantel-Cox] test, chi-squared P≦0.01) in WT mice after IR injury. Data represent the mean6SEM. *P≦0.05; **P≦0.01.

FIG. 8A-8B. (A) Percentage of CD11b in normal CD4-KEAP1-KO and KEAP1fl/fl mice kidneys. (B) Percentage of CD11c in normal CD4-KEAP1-KO and KEAP1fl/fl mice kidneys.

FIG. 9A-9B. (A) Percentage of Foxp3 positive cells in the lymph node (LN) and thymus of normal CD4-KEAP1-KO and KEAP1fl/fl mice. (B) Percentage of CD4, CD8, DNT and double positive cells in thymus from normal CD4-KEAP1-KO and KEAP1fl/fl mice were comparable.

FIG. 10. Confirmation of AKI, 24 h after IRI, by SCr in mice sacrificed for flow cytometric analysis.

FIG. 11. Ex-vivo activation of purified T cell with CDDO-Im resulted in significant increase in Nrf2 target gene expression in purified T cells in mice and human PBMCs. To prove whether ex-vivo Nrf2 activation of T cell can achieved ex-vivo we isolated T cell from 6-8 wk old male wild type mice using Pan T cell isolation kit and were treated approximately 1 million pure T cells with two different concentrations (20 nM and 50 nM) of well-known Nrf2 activator, CDDO-Im, for 24 hours. RNA isolated from these cells was analyzed by quantitative real-time PCR for expression of Nrf2 target genes, NQO1, HO-1 and GCLM. We found a significant increase in NQO1 and HO-1 mRNA expression following CDDO-Im treatment. Furthermore, we treated peripheral blood mononuclear cells (PBMCs) from healthy individuals and treated them with CDDO-Me (Bardoxolone), an Nrf2 activator related to triterpenopid derivative CDDO-Im. We observed a significant increase in the gene expression of NQO1 (p=0.02), HO-1 (p=0.05) and GCLM (p=0.01) following CDDO-Me treatment.

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

“Gene targeting” is a type of homologous recombination that occurs when a fragment of genomic DNA is introduced into a mammalian cell and that fragment locates and recombines with endogenous homologous sequences.

A “knockout mouse” (or “KO mouse”) is a mouse in the genome of which a specific gene has been inactivated by the method of gene targeting. A knockout mouse can be a heterozygote (i.e., one defective/disrupted allele and one wild-type allele) or a homozygote (i.e., two defective/disrupted alleles). “Knockout” of a target gene means an alteration in the sequence of the gene that results in a decrease or, more commonly, loss of function of the target gene, preferably such that target gene expression is undetectable or insignificant. A knock-out of a KEAP1 gene means that function of the KEAP1 gene has been substantially decreased or lost so that KEAP1 expression is not detectable (or may only be present at insignificant levels). The term “knockout” is intended to include partial or complete reduction of the expression of at least a portion of a polypeptide encoded by the targeted endogenous gene of a single cell, a population of selected cells, or all the cells of a mammal.

KO mice of the present invention include “conditional knockouts” in which, by inclusion of certain sequences in or surrounding the altered target, it is possible to control whether or not the target gene is rendered nonfunctional. This control can be exerted by exposure of the animal to a substance that promotes target gene alteration, introduction of an enzyme that promotes recombination at the target gene site (e.g., Cre in the Cre-lox system), or any other method that directs or controls the target gene alteration post-natally. Conditional knock-outs of KEAP1 gene function are also included within the present invention. Conditional knock-outs are transgenic animals that exhibit a defect in KEAP1 gene function upon exposure of the animal to a substance that promotes target gene alteration, introduction of an enzyme that promotes recombination at the target gene site (e.g., Cre in the Cre-loxP system), or other method for directing the target gene alteration. For example, an animal having a conditional knock-out of KEAP1 gene function can be produced using the Cre-loxP recombination system (see, e.g., Kilby et al. 1993 Trends Genet 9:413-421). Cre is an enzyme that excises the DNA between two recognition sequences, termed loxP. This system can be used in a variety of ways to create conditional knock-outs of KEAP1. For example, in addition to a mouse in which the KEAP1 sequence is flanked by loxP sites a second mouse transgenic for Cre is produced. The Cre transgene can be under the control of an inducible or developmentally regulated promoter (Gu et al. 1993 Cell 73:1155-1164; Gu et al. 1994 Science 265:103-106), or under control of a tissue-specific or cell type-specific promoter (e.g., a kidney-specific promoter). The KEAP1 transgenic is then crossed with the Cre transgenic to produce progeny deficient for the KEAP1 gene only in those cells that expressed Cre during development.

In certain embodiments, the methods of the present invention are used in the treatment of AKI. In particular embodiments, T-cells from the patient are obtained and treated in vitro to activate Nrf2 activity. The terms “Nrf2 activator” and “Nuclear factor (erythroid-derived 2)-like 2 activator” as used herein relate to chemical compounds or elements that increase the activity of Nrf2. There are various Nrf2 activators known in the art which are suitable for use in the present invention including, but not limited to, tert-butylhydroquinone (tBHQ); sulforaphane; Oltipraz (4-methyl-5-(2-pyrazinyl)-3-dithiolethione); bardoxolone methyl (also known as CDDO-Me or RTA 402) from Reata pharmaceuticals; dihydro-CDDO-trifluoroethyl amide (dh404); resveratrol; anethole dithiolethione; 6-methylsulphinylhexyl isothiocyanate; curcumin; caffeic acid phenethyl ester; and 4′-bromoflavone. Other Nrf2 activators include, but are not limited to, 1,2,3,4,6-Penta-O-Galloyl-Beta-D-Glucose; 1,2-Diphenol (Catechol); 1,2-Dithiole-3-Thione; 1,4-Diphenols (P-Hydroquinone); 1-[2-Cyano-3-,12-Dioxooleana-1,9(11)-Dien-28-Oyljlmidazole (CDDO-Imidazol); 15-Deoxy-12,14-Pgj2; 1-Chloro-2,4-Dinitrobenzene; 2,3,7,8-Tetrachlorodibenzo-P-Dioxin; 2-Cyano-3,12-Dioxooleana-1,9(11)-Dien-28-Oic Acid (CDDO); 2-Indol-3-Yl-Methylenequinuclidin-3-Ols; 3-Hydroxyanthranilic Acid; 3-Methylcholanthrene; 4-Hydroxyestradiol; 4-Hydroxynonenal; 6-Methylsulfinylhexyl; Isothiocyanate; 9-Cis-Retinoic Acid; Acetaminophen; Acetylcarnitine; Acrolein; Allyl Isothiocyanate; Alpha-Lipoic Acid; Apomorphine; Arsenic; AUR ((2,3,4,6-Tetra-O)-Acetyl-1-Thio-D-Glucopyranosato-S)(Triethylphosphine) Gold(I); Autg ((1-Thio-D-Glucopyranosato) Gold(I); Autm (Sodium Aurothiomalate); Bis(2-Hydroxybenzylidene)Acetone; Bleomycin; B-Naphthoflavone; Broccoli Seeds; Bucillamine; Butein; Butylated Hydroxyanisole; Butylated Hydroxytoulene; Cadmuim Chloride; Cafestol; Carbon Monoxide; Carnosol; Catechol; chalcones (1,3-Diphenyl-2-propen-1-ones); chalcone derivatives (such as those described in Kumar, et al., J Med Chem, 54:4147-59 (2011) and Yang et al., Free Rad Biol Med, 51:2073-2081(2011), the disclosures of each of which are hereby incorporated by reference herein); Chlorogenic Acid; Cigarette Smoke; Cobalt (Cobalt Chloride); Copper; Coumarin; Curcumin; Deprenyl (Selegiline); Dexamethasone 21-Mesylate; Diallyl Disulfide; Diallyl Sulfide; Diallyl Trisulfide (DATS); Diesel Exhaust; Diethylmaleate; Epicatechin-3-Gallate; Epigallocatechin-3-Gallate; Eriodictyo; Ferulic Acid (Trans-4-Methoxycinnamic Acid, 99% Purity); Fisetin; Flunarizine; Gallic Acid (3,4,5-Trihydroxybenzoic Acid); Gentisic Acid; Glucose Oxidase; Glycosides From Digitalis Purpurea; Heme; Hemin; Hydrogen Peroxide; Hyerpoxia; Indole-3-Carbinol; Indomethacin; Insulin; Iodoacetic Acid; Kahweol Palmitate; Laminar Flow; Lead; Limettin (LMTN); Lipoic Acid; Lipopolysacharide; Luteolin; Lycopene; Menadione; Mercury; Nickel (II); Nitric Oxide-Donating Aspirin; Oxidized Low-Density Lipoproteins; Paraquat; Parthenolide; P-Coumaric Acid (Trans-4-Hydroxycinnamic Acid); Phenethyl Isothiocyanate; Phloretin Phorbol 12-Myristate 13-Acetate (PMA); P-Hydroxybenzoic Acid; Proteasome Inhibitor MG-132; Proteasome Inhibitors (Lactacystin Or MG-132); Pyrrolidine Dithiocarbamate; Quercetin; Quercetin 3-O-Beta-L-Arabinopyranoside; Sodium Arsenite; Spermidine; Spermine; Spermine Nonoate; TNF-Alpha; Trans-Stilbene Oxide; Triterpenoid-155; Triterpenoid-156; Triterpenoid-162; Triterpenoid-225; Tunicamycin; Ultraviolet A; Irradiation; Wasabi Extract; Xanthohumol (XH); Zerumbone; Zinc; Patulin; Methosyvone; Dehydrovariabilin; Biochanin A; Pdodfilox; 8-2′-Dimethoxyflavone; 6,3′-Dimethoxyflavone; Pinosylvin; Gentian Violet; Gramicidin; Thimerosal; Cantharidin; Fenbendazole; Mebendazole; Triacetylresveratrol; Resveratrol; Tetrachloroisopthalonitrile; Simvastatin; Valdecoxib; beta-Peltatin; 4,6-Dimethoxy-5-methylsioflavone; Nocodazole; Pyrazinecarboxamide; (+)-thero-1-Phenyl-2-decanoylamino-3-morpholino-1-propanol hydrochloride; SU4132. Additional examples of Nrf2 activators can be found in U.S. Published Patent Application 2011/0250300 to Biswal et al. and U.S Published Patent Application 2004/0002463 to Honda et al., the disclosures of each of which are hereby incorporated by reference herein.

Also included among useful Nrf2 activators are pharmaceutically acceptable molecular conjugates or salt forms of the activators described above, that maintain activity as Nrf2 activators as defined herein. Examples of pharmaceutically acceptable salts of Nrf2 activators include sulfate, chloride, carbonate, bicarbonate, nitrate, gluconate, fumarate, maleate, or succinate salts. Other embodiments of pharmaceutically acceptable salts contain cations, such as sodium, potassium, magnesium, calcium, ammonium, or the like. Other embodiments of useful Nrf2 activators are hydrochloride salts. For providing enhanced cell permeability to an Nrf2 activator moiety, various conjugated forms are useful, e.g., Nrf2 activator-lipid conjugates, emulsified conjugates of Nrf2 activators, lipophillic conjugates of Nrf2 activators, and liposome- or micelle-conjugated Nrf2 activators. (Fenske, D B et al., Biochim Biophys Acta, 2001:1512(2):259-72; Khopade, A J et al., Drug Deliv. 2000: 7(2): 105-12; Lambert, D M et al., Eur. J. Pharm. Sci. 2000: 11 Suppl 2:S15-27; Pignatello, R et al., Eur J Pharm Sci. 2000: 10(3):237-45; Allen, C et al., Drug Deliv. 2000: 7(3):139-45; Dass, C R et al., Drug Deliv. 2002: 9(1): 11-8; Dass, C R, Drug Deliv. 2000:7(3): 161-82; which are hereby incorporated by reference herein). The Nrf2 activators can be synthesized by known chemical means or can be procured commercially.

Ischemia reperfusion (IR)-induced AKI is associated with significant mortality in native kidneys and worsens outcomes after kidney transplantation. Excessive oxidative stress, apoptosis, epithelial and endothelial cell dysfunction, and inflammation are among the important pathophysiologic mechanisms during ischemic AKI. Recent work demonstrates an important pathophysiologic role for T lymphocytes in AKI, but the underlying mechanisms are poorly understood. Traditional mechanisms of immune activation and responses through allo- or self-antigen are not known to occur during AKI. Some data suggest that excessive oxidative stress, such as during IR injury, can either activate various subsets of T cells or reduce T cell function and compromise T cell receptor (TCR) signaling. However, the role of oxidative stress involvement in T cells during AKI is unknown, as is the effect of T cell-specific augmentation of antioxidant responses.

Studies on mechanisms of AKI from a number of teams demonstrate an important role for Nrf2, a transcription factor that regulates the expression of multiple antioxidant and phase II metabolism genes. Nrf2 is a key mediator that mitigates both ischemic and nephrotoxic AKI, as well as various other oxidative stress-driven diseases. To date, Nrf2 has been shown to work in AKI through effects on resident renal epithelial cells. The transcriptional activity of Nrf2 is regulated by kelch-like ECH associated protein 1 (Keap1), which retains Nrf2 in the cytoplasm and promotes its proteolytic degradation. We therefore hypothesized that T cell-specific Nrf2-mediated signaling was an important converging mechanism by which both T cells and Nrf2 regulate AKI. To test this hypothesis, we generated mice with genetic deletion of Keap1 using a T cell-specific Cre-loxP recombination strategy.

Our data demonstrate that T cell-specific activation of Nrf2 increases the baseline frequency of kidney CD25+Foxp3+ regulatory T cells (Tregs) and significantly attenuates pro-inflammatory cytokine production by CD4+ T lymphocytes in the kidney. Furthermore, mice with high Nrf2 in T cells had fewer CD11b+CD11c+ and F4/80+ cells in their kidneys. The high Nrf2 activity in T cells resulted in significant structural and functional protection against IR-induced AKI. Furthermore, T cells with activated Nrf2 were effective as cell therapy for AKI when adoptively transferred to wild-type (WT) mice. These results demonstrate a novel mechanism by which T cells mediate AKI and reveal an unexpected cell type by which Nrf2 modulates acute tissue injury.

### Materials and Methods

Generation and Characterization of CD4-Keap1-KO Mice.

T cell-specific Keap1-deficient (referred to as CD4-Keap1-KO) mice were generated by crossbreeding Keap1F/F mice with CD4-Cre mice. Keap1 floxed mice used for these studies were kindly provided by Dr. Shyam Biswal and have been completely characterized. CD4-Cre mice were purchased from Taconics (Hudson, N.Y.). In these CD4-Cremice, the transgene is under the control of the CD4 promoter/enhancer/silencer, which first allows expression of CD4-Cre in thymocytes at the double-positive (CD4+CD8+) stage. The silencer region extinguishes transgene expression at the DN (CD42CD82) stage as well as in the CD42CD8+ stage. The mice were genotyped to confirm the presence of the Cre transgene, flox status, and deletion of Keap1 exons 2 and 3 with PCR using primer sets shown in Table 1.

TABLE 1
Primer Information for PCR Based Confirmation of
CRE, KEAP1 Floxed and KEAP1 Deleted Allele Status

| | Primer | | Product |
|---|---|---|---|
| S No. | Name | Sequence 5′-3/ | Size |
| 1 | Generic | GCG GTC TGG CAG TAA AAA | 100 bp |
| | CRE FP | CTA TC | |
| | | (SEQ ID NO: 1) | |
| | Generic | GTG AAA CAG CAT TGC TGT | |
| | CRE RP | CAC TT | |
| | | (SEQ ID NO: 2) | |
| 2 | KEAP1flox | CGA GGA AGC GTT TGC TTT | KEAP |
| | FP | AC | floxed |
| | | (SEQ ID NO: 3) | allele: |
| | KEAP1flox | GAG TCA CCG TAA GCC TGG | 383 bp |
| | RP | TC | |
| | | (SEQ ID NO: 4) | |
| 3 | KEAP1 | GAG TCC ACA GTG TGT GGC C | Deleted |
| | deletion | (SEQ ID NO: 5) | allele: |
| | FP | | 288 bp |
| | KEAP1 | GAG TCA CCG TAA GCC TGG | Wild type |
| | deletion | TC | allele: |
| | RP | (SEQ ID NO: 6) | 2954 Kb |

Mouse Model of AKI.

An established mouse model of renal IR injury was used. All animal experiments were performed using Johns Hopkins University Institutional Animal Care and Use Committee-approved protocols. Animals were anesthetized with sodium pentobarbital (Voshell's Pharmacy, Baltimore, Md.) at a dose of 75 mg/kg (intraperitoneal injection). The mice were put on a heating pad (45° C.) during the procedure and core body temperature was maintained at approximately 37° C. Left and right renal pedicles were bluntly dissected after laparotomy and ischemia was induced by placing a nontraumatic microvascular clip (Roboz, Gaithersburg, Md.) on each renal pedicle for 30 minutes. During the procedure, mice were well hydrated by infusing warm saline (37° C.-40° C.) directly into the peritoneal cavity. The kidneys were allowed to reperfuse by removing the microvascular clips, wounds were sutured, and animals were allowed to recover on the heating pad. Once awake, the mice were transferred to a clean cage and housed in the animal facility at room temperature with food and water ad libitum.

Assessment of Renal Function.

Blood samples were obtained from the tail before (0 hours) and 24, 48, and 72 hours after kidney IR injury to collect serum. SCr was measured as a marker of renal function by a Cobas Mira Plus automated analyzer system (Roche) by using creatinine measurement reagents (Pointe Scientific Inc., Canton, Mich.).

Histologic Evaluation of Kidney Injury.

Upon euthanasia, the kidneys were harvested and cut into three equal transverse pieces. One piece from each kidney was fixed with 10% buffered formalin phosphate and embedded with paraffin for histologic evaluation. The remaining two kidney pieces were either snap-frozen with liquid nitrogen or stored in RNAlater solution (Life Technologies, Grand Island, N.Y.) for molecular studies. Tissue sections (5 mm) were stained with hematoxylin and eosin. A renal pathologist (L.C.R.) at Johns Hopkins Hospital, who was blinded to the experimental groups, scored the percentage of necrotic tubules out of total tubules in each of at least 10 high-power fields in the cortex and outer medulla, and the average percentage of tubular necrosis in all fields was presented as the renal tubular injury score of each mouse.

Antioxidant Gene Expression Analyses.

Total RNA (1 mg) from purified T cells was isolated with the RNeasy mini kit (Qiagen, Valencia, Calif.) and reverse transcribed using a high capacity cDNA synthesis kit (Life Technologies). A gene-specific TaqMan primer and probe sets were used to assess transcriptional status of Nrf2, Nqo1, Ho-1, Gclm, and Gcic in Quantstudio 12K flex real-time PCR (Life Technologies). The absolute expression values for each gene were normalized to that of b-actin and the relative gene expression values calculated.

Assessment of Kidney Inflammation.

Levels of IFN-g, TNF-a, monocyte chemoattractant protein-1, and IL-10 were assessed by the Bio-Plex multiple cytokine kit (Bio-Rad, Hercules, Calif.) to evaluate inflammation of kidney tissue. The total protein concentration of each sample was determined using a bicinchoninic acid protein assay kit (Thermo Fisher Scientific, Rockford, Ill.) and was used to normalize the measured cytokine levels.

Phenotypic Characterization and Intracellular Cytokine Analyses.

Phenotypic characterization and intracellular cytokine analysis in kidney-infiltratingCD45+Tcells, inguinal LN lymphocytes, and thymocytes were performed in normal and post-ischemic mice. The following fluorochrome-conjugated mAbs to mouse antigens were used to construct four different panels (Table 2) for flow cytometry analysis: CD45-APC-Cy7 (BioLegend, San Diego, Calif.), TCR-Pacific Blue/APC (Invitrogen/BioLegend), CD4-PE-Cy7/PE (BioLegend/BD Biosciences, Franklin Lakes, N.J.), CD8-PerCP (BioLegend), CD25-APC (eBioscience, San Diego, Calif.), CD19-APC(BioLegend), NK1.1-APC(eBioscience), Foxp3-PE (eBioscience), IFN-g-PE (BD Biosciences, Franklin Lakes, N.J.), F4/80+-PE (eBioscience), CD69-FITC (BD Biosciences), CD11b+-FITC (BD Biosciences), TNF-a-FITC (BD Biosciences), Ly-6G(Gr1)-FITC (eBioscience), CD11c+-PE-Cy7 (eBioscience), and IL-17-BV-605 (BioLegend).

Briefly, kidney mononuclear cells were isolated using density gradient centrifugation (Percoll) as previously described 55 and CD45+ cells were enriched using CD45 microbeads (Miltenyi Biotech, San Diego, Calif.). Freshly isolated lymphocytes from the kidney (approximately 53106 cells) LN (13106 cells), and thymus (13106 cells) were stimulated with PMA (5 ng/ml) and ionomycin (500 ng/ml) before staining for surface markers and intracellular cytokines. Labeled samples were analyzed with the LSRII flow cytometer (BD Biosciences). Controls (fluorescence minus one) were used to correctly identify and gate cell populations during analysis using FlowJo software (TreeStar Inc., Ashland, Oreg.).

TABLE 2
Antibody Panel Used For Phenotypic Characterization
And Intracellular Cytokine Analysis.

| Panel 1 | Panel 2 | Panel 3 | Panel 4 | FMO A | FMO B | FMO C |
|---|---|---|---|---|---|---|
| CD69 | CD19 | IFN-γ PE | F4/80 PE | | | |
| FITC | APC | | | | | |
| FOXP3 | CD11C | TNF-α | Ly-6(Gr- | | | |
| PE | PE CY7 | FITC | 1) FITC | | | |
| CD25 | CD11B | IL-17 BV | NKT APC | | | |
| APC | FITC | 605 | | | | |
| CD8 | CD8 | CD8 | CD8 | CD8 | CD8 | CD8 |
| PERCP | PERCP | PERCP | PERCP | PERCP | PERCP | PERCP |
| CD4 PE | CD4 PE | CD4 PE | CD4 PE | CD4 PE | CD4 PE | CD4 PE |
| CY7 | | CY7 | CY7 | CY7 | | CY7 |
| TCR P. | TCR P. BLUE | TCR APC | TCR P. | TCR P. | TCR P. | TCR APC |
| BLUE | | | BLUE | BLUE | BLUE | |
| CD45 | CD45 | CD45 | CD45 | CD45 | CD45 | CD45 |
| APC CY7 | APC CY7 | APC CY7 | APC CY7 | APC CY7 | APC CY7 | APC CY7 |

FMO pertaining to each panel was used to gate proper cell populations during analysis.

CD4+ T Cell Activation In Vitro.

CD4+ T cells were isolated with the CD4+ T cell isolation kit (Miltenyi Biotech). Briefly, approximately 13106 cells/ml per well were plated in a 24-well plate pre-coated with CD3/CD28 (1 mg/ml) and IL-2 (20 IU/ml). At days 3 and 7, intracellular levels of IFN-g and IL-4 were analyzed by flow cytometry as described earlier.

Adoptive Transfer of T Cells.

T cells were isolated from mouse spleen using the Pan-T cells isolation kit (Miltenyi Biotech) and approximately 153106 T cells were adoptively transferred to WT C57B1/6 mice (The Jackson Laboratory, Bar Harbor, Me.) by tail vein injection. T cells were CFSE labeled to confirm the success of tail vain injection. Twenty-four hours after T cell transfer, the mice underwent IR-induced AKI. The presence of transferred CFSE labeled T cells was confirmed in recipient blood before IR surgery.

Immunoblotting.

T cells were isolated from CD4-Keap1-KO (n=3) and Keap1F/F mice (n=3) as described earlier, and nuclear and cytoplasmic extracts were prepared using the NE PER kit (Thermo Fisher Scientific). For Western blot analysis, a total of 50 mg cytoplasmic extract and 20 mg nuclear extract from each sample were separated on a 10% SDS-PAGE, and the membranes were probed with antibodies specific for Nrf2 (Santa Cruz Biotechnology, Santa Cruz, Calif.) andNqol (NeoBioLab, Woburn, Mass.). b-actin (Sigma-Aldrich, St. Louis, Mo.) and Lamin B (Santa Cruz Biotechnology) were used as the loading controls. The blots were developed using an enhanced chemiluminescence kit (HyGlo; Denville Scientific Inc., Metuchen, N.J.) and band intensities were measured using ImageJ Software (National Institutes of Health, Bethesda, Md.).

Statistical Analyses.

Data are presented as the mean 6SEMor SD, and are compared by a paired, two-tailed t test for a single comparison between two groups. Cumulative survival was analyzed by the log-rank (Mantel-Cox) test. Statistical significance of difference was defined as a P value #0.05.

### Results

T Cell-Specific Deletion of Keap1 Increases Basal Antioxidant Response.

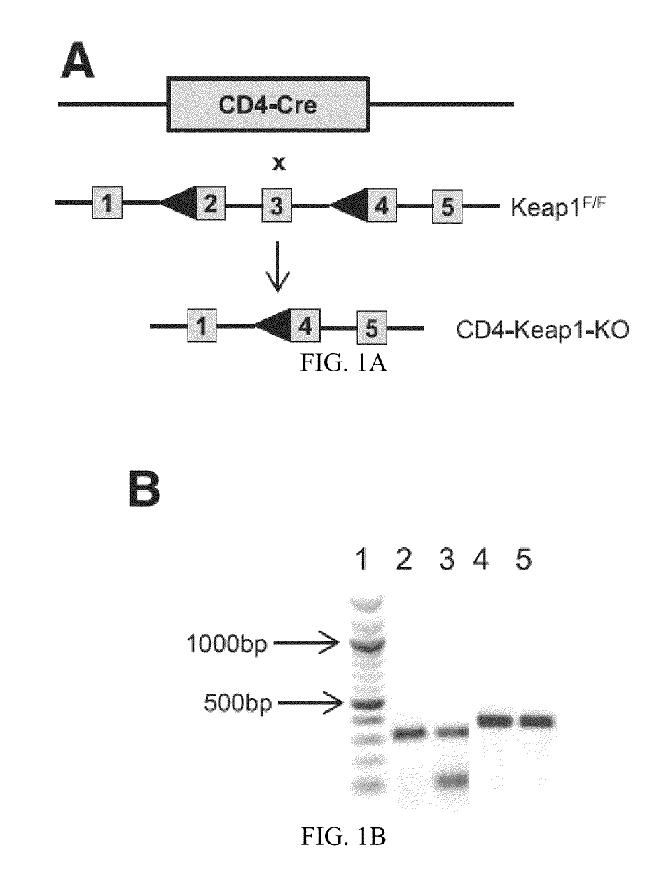

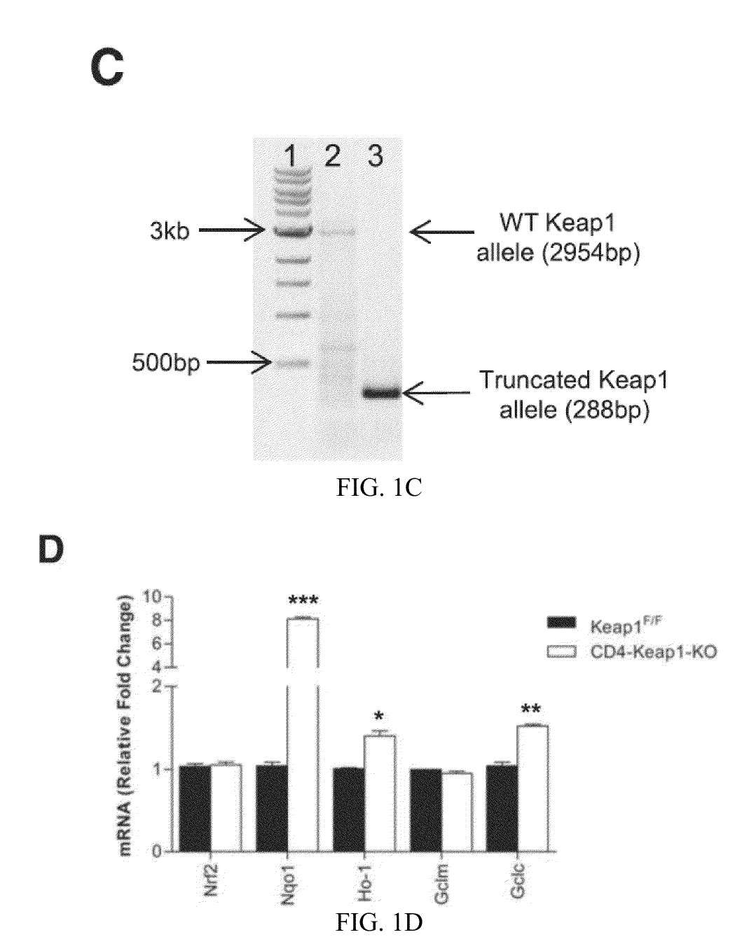

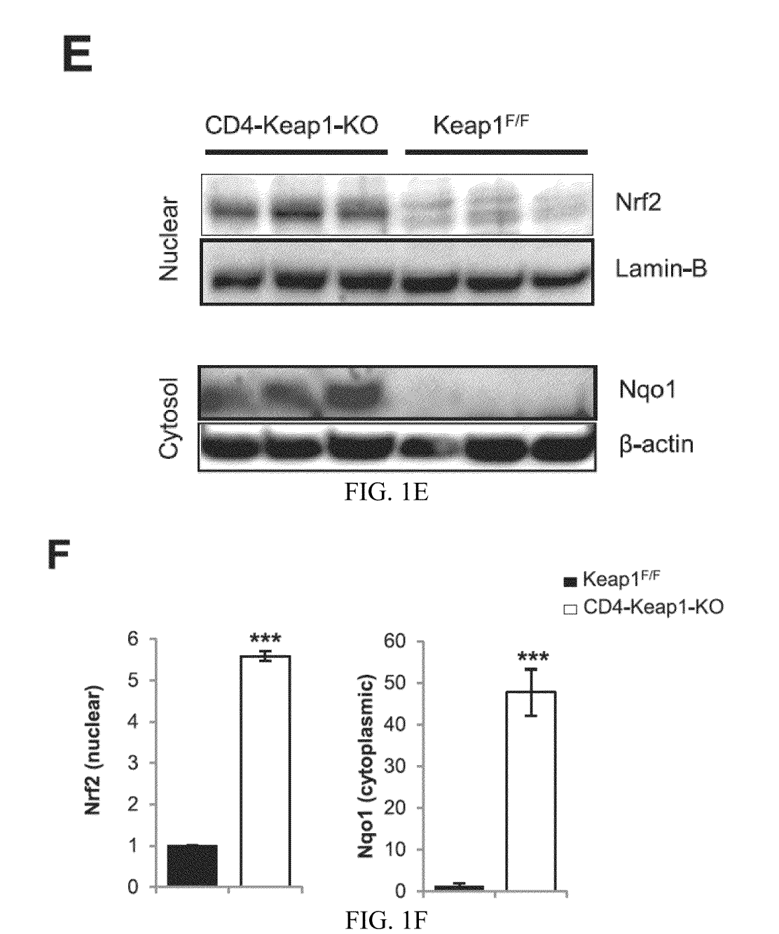

To examine the role of Nrf2-regulated antioxidant response in T cells and its effect on ischemic AKI, we genetically deleted Keap1 from T cells by breeding mice with the lox-P allele of Keap1 (Keap1F/F) with CD4-Cre mice (FIG. 1A). The CD4-Cre transgene brings about selective deletion of genes flanked by the lox-P sequence in thymocytes at the double-positive (CD4+CD8+) stage. The Cre is silenced in doubled negative (DN) (CD42CD82) and CD42CD8+ mature thymocytes.17 This strategy resulted in the generation of Keap1F/FCD4-Cre mice (hereafter referred to as CD4-Keap1-KO) with successful deletion of exons 2 and 3 of Keap1, mostly in CD4+ and CD8+ T cells (FIGS. 1, B and C). We observed no signs of physiologic or phenotypic abnormalities due to this deletion in these mice. To assess the effect of Keap1 deletion on Nrf2 activity, we measured the expression of Nrf2-regulated antioxidant genes in purified T cells from the spleen by real-time PCR. Purified T cells from CD4-Keap1-KO mice showed significantly higher Nqo1 (P≦0.01), Ho-1 (P=0.05), and Gcic (P≦0.01) mRNA expression at baseline compared with Keap1F/F mice (FIG. 1D). There was no difference in the expression of Nrf2 mRNA between CD4-Keap1-KO and Keap1F/F mice, indicating that disruption of Keap1 does not affect Nrf2 transcription. Furthermore, Keap1 deletion significantly increased (P≦0.001) nuclear Nrf2 and cytoplasmicNqo1 protein levels in T-cells isolated from CD4-Keap1-KO mice compared with Keap1F/F mice (FIGS. 1, E and F). The effect of Keap1 deletion on antioxidant gene expression in this study is corroborated by previous studies in non-T cell models.

Nrf2 Augmentation Affects Immune Cell Recruitment, Activation, and Intracellular Cytokine Production by T Cells.

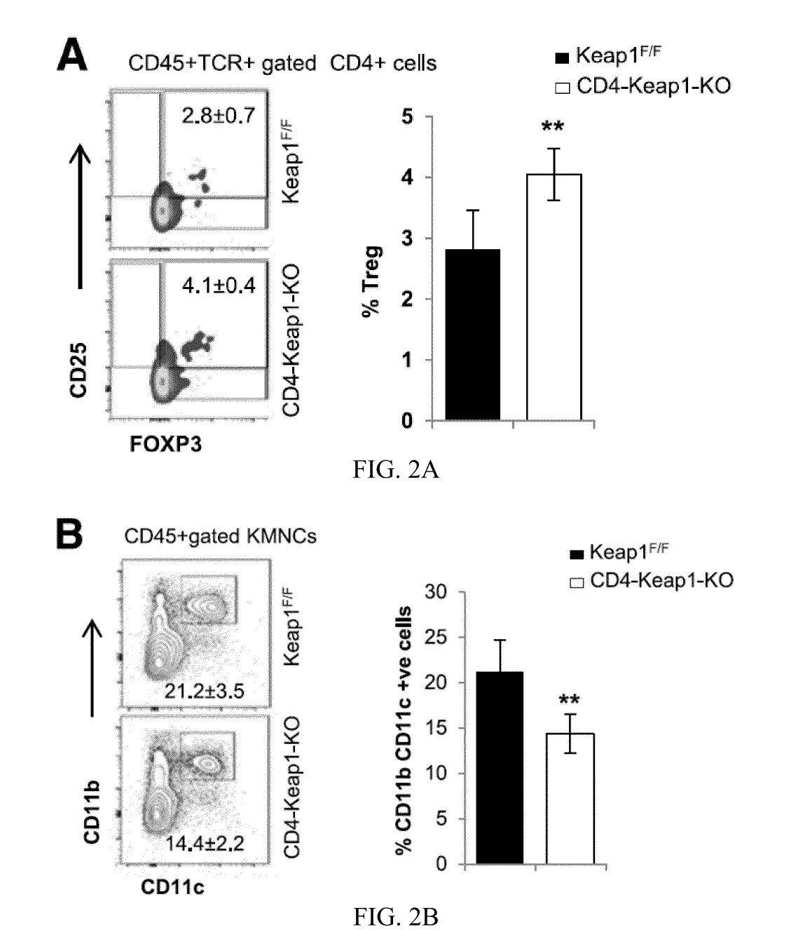

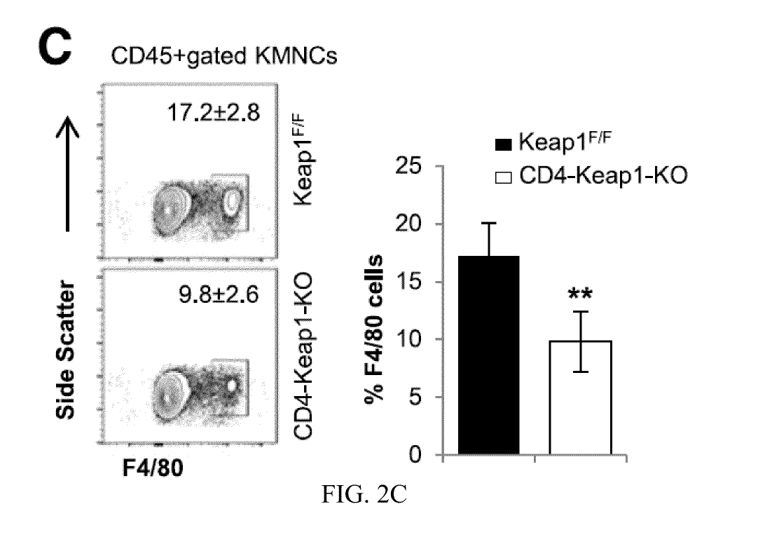

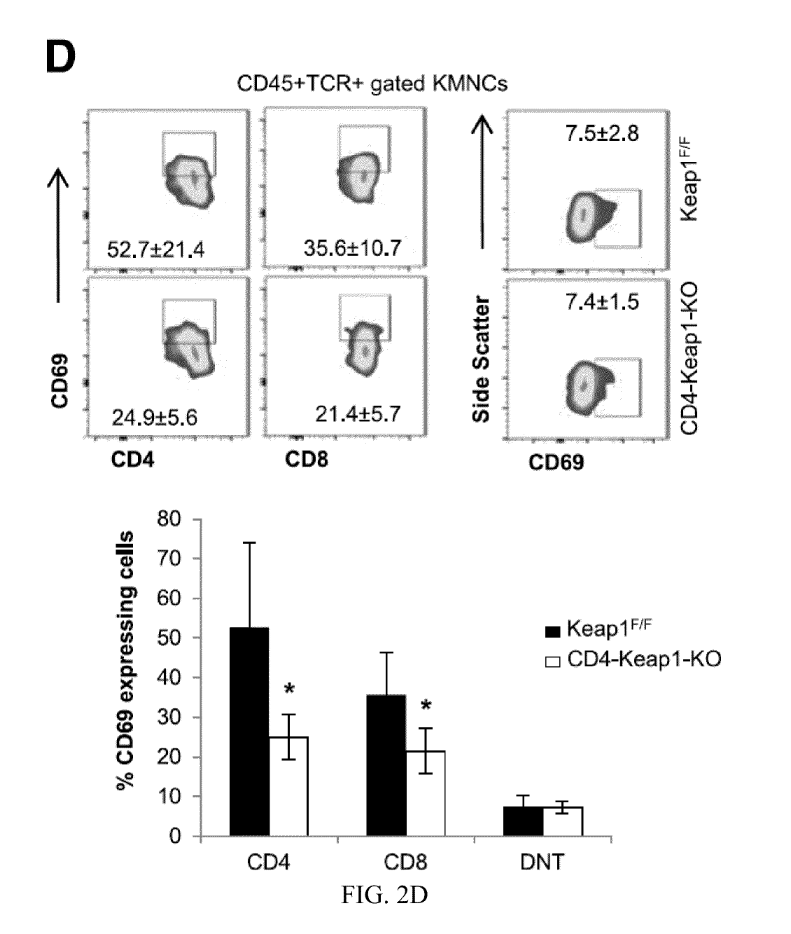

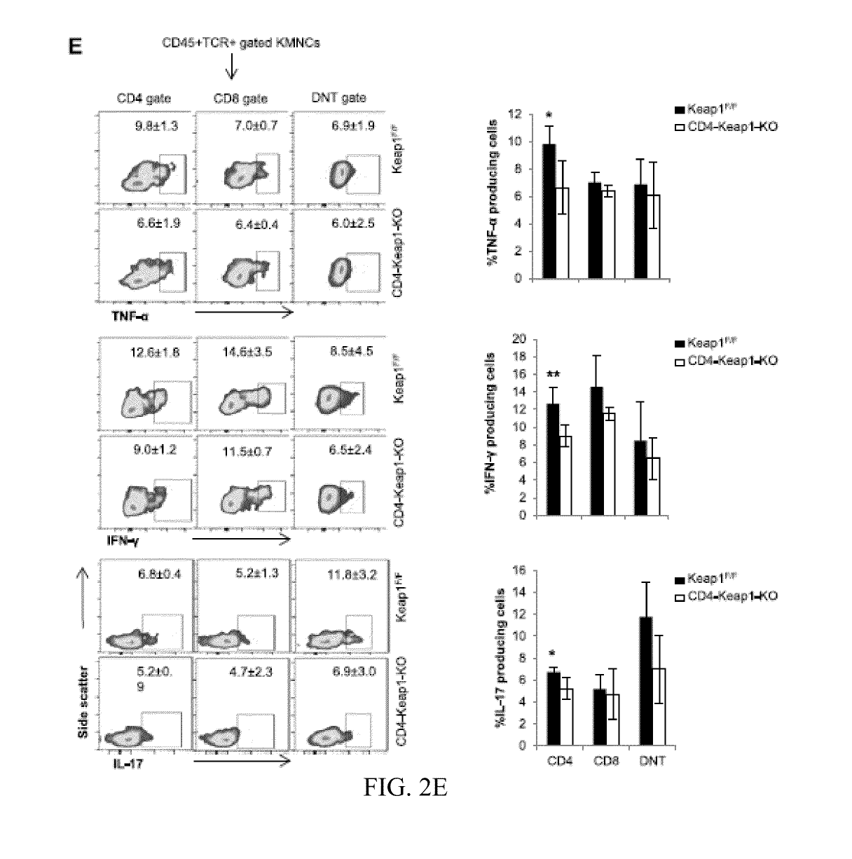

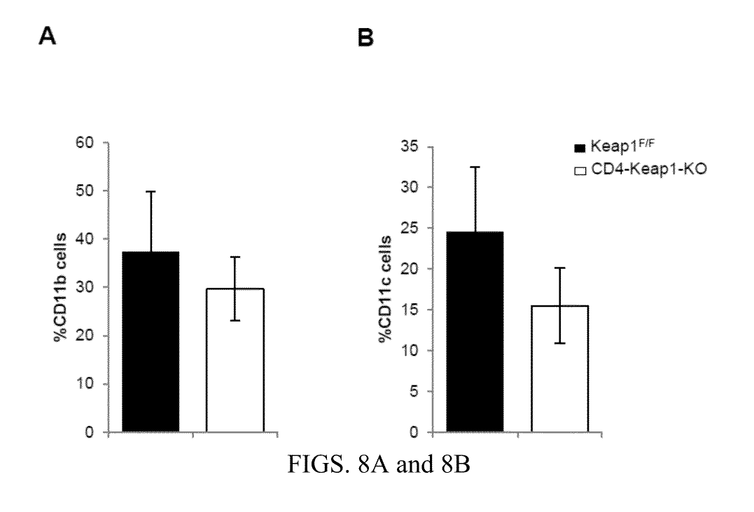

We further compared phenotypic changes, activation status, and cytokine production in CD45+TCR+ cells isolated from the kidney, inguinal lymph node (LN), and thymus of CD4-Keap1-KO and Keap1F/F mice at baseline (no IR). Although CD4-Cre is expressed at the CD4+CD8+ stage, we included CD42CD82 (DN) T cells in our analysis because they represent a major component of normal and ischemic kidneys. 20, 21 Furthermore, some of them may be derived from reverting CD4+CD8+ T cells. 22, 23 Flow cytometric analysis of CD45+TCR+CD4+ cells revealed a significantly higher percentage of CD25+Foxp3+ Tregs in kidneys of CD4-Keap1-KO mice compared with Keap1F/F mice (4.1%6 0.4% versus 2.8%60.7%; P=0.02) (FIG. 2A). Furthermore, the percentage of CD11b+CD11c+ dendritic cells (DCs) (14.4%62.2% versus 21.2%63.5%; P=0.01) and F4/80+ macrophages (9.8%6 2.6% versus 12.2%62.8%; P≦0.01), among total CD45+ kidney mononuclear cells from baseline kidneys of CD4-Keap1-KO mice, was significantly lower compared with Keap1F/F controls (FIGS. 2, B and C, respectively). Percentages of CD11b+(29.7%66.6% versus 37.3%612.5%) and CD11c+(15.5%64.6% versus 24.6%67.9%) cells were not different between CD4-Keap1-KO kidneys and Keap1F/F mice (FIG. 8). We further examined the expression of CD69 to assess activation status of CD4, CD8, and double negative (DN) T cells isolated from kidneys of CD4-Keap1-KO and Keap1F/F mice (FIG. 2D). We observed significantly lower CD69 expression in CD4 (24.9%65.6% versus 52.7%621.4%; P=0.04) and CD8 (21.4%6 5.7% versus 35.6%610.8%; P=0.05) cells from CD4-Keap1-KO kidneys compared with Keap1F/F counterparts. Percentage of CD69+DNT cells were comparable (7.4%6 1.5 versus 7.5%62.6) between CD4-Keap1-KO and Keap1F/F mice.

We then studied the effect of Nrf2 activation on pro-inflammatory cytokine production by renal T cells by assessing intracellular levels of TNF-a, IFN-g, and IL-17 in CD4, CD8, and DNT cells isolated from CD4-Keap1-KO and Keap1F/F kidneys (FIG. 2E). Baseline levels of intracellular TNF-a (6.6%61.9% versus 9.8%61.3%; P=0.03), IFN-g (9.0%6 1.2% versus 12.6%61.8%; P=0.01), and IL-17 (5.2%60.9% versus 6.8%60.4%; P=0.02) were significantly lower in CD4 T cells from CD4-Keap1-KO mice than in Keap1F/F mice. We observed a similar trend in CD8 and DNT cells isolated from CD4-Keap1-KO mice kidneys but those changes were not statistically significant.

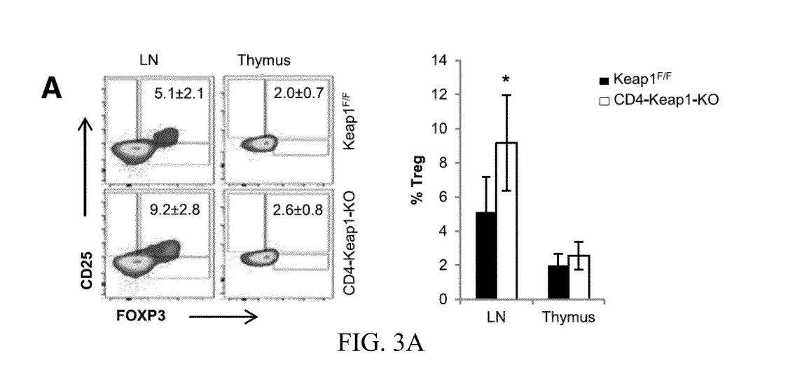

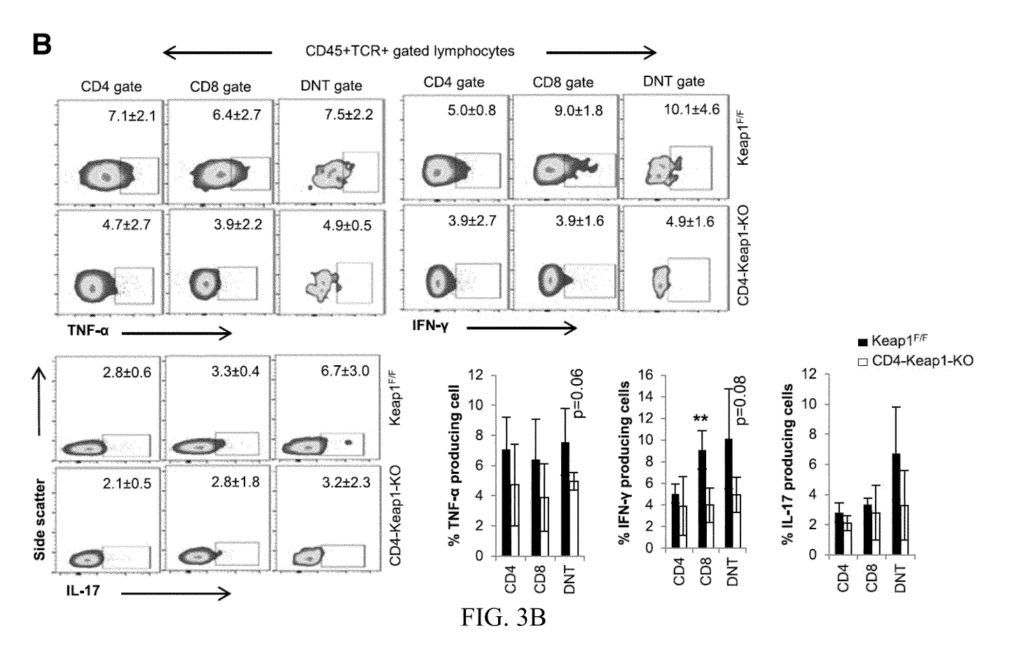

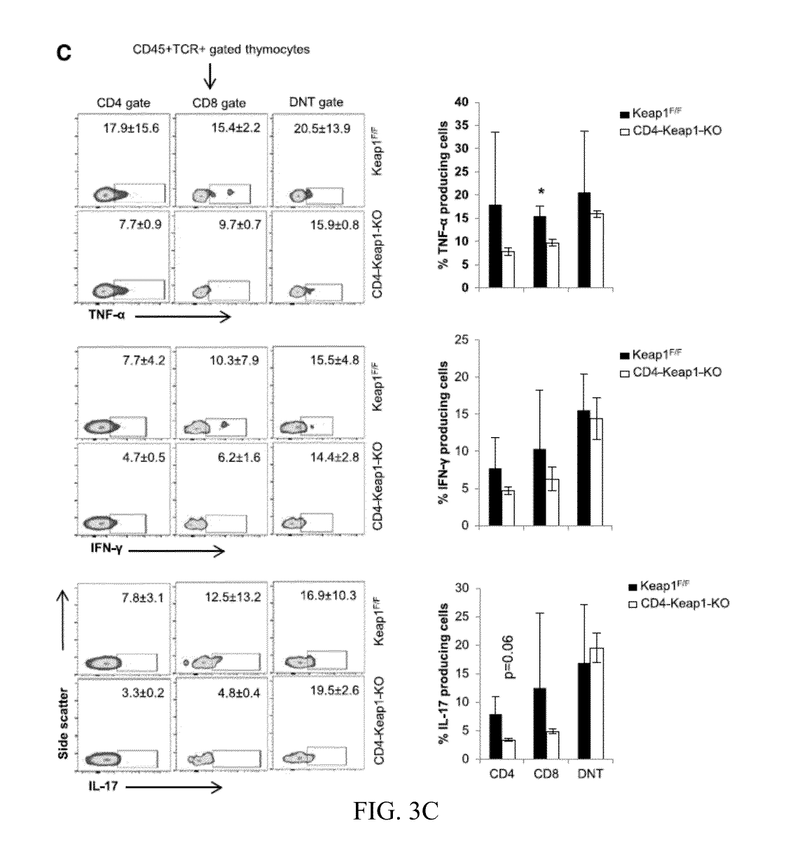

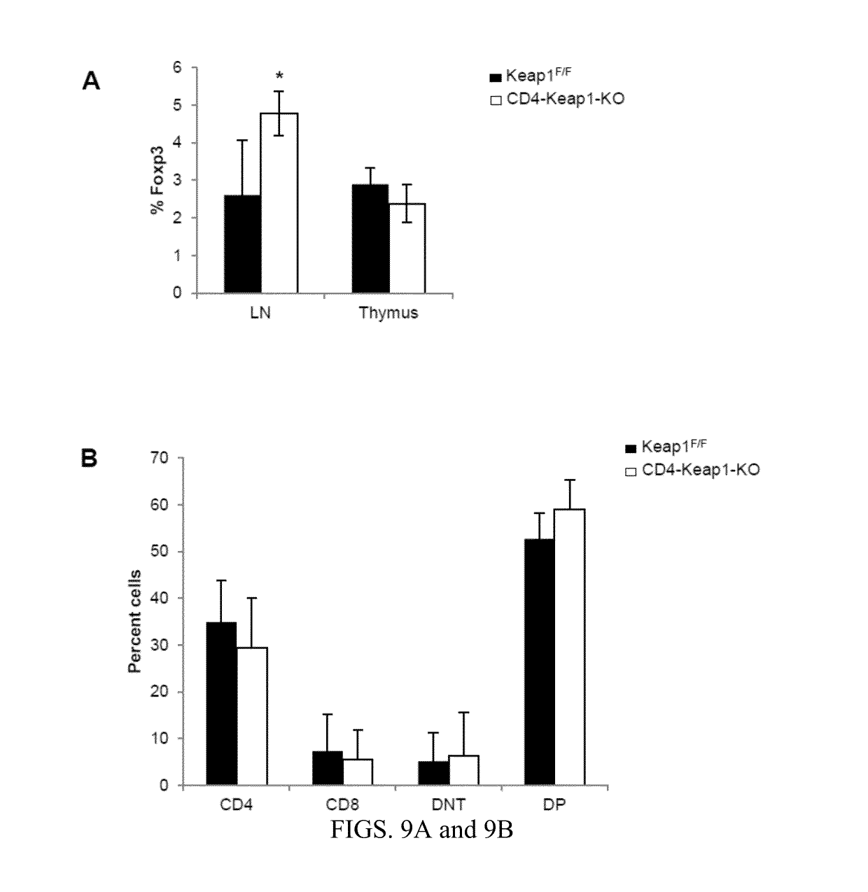

The frequency of CD4+CD25+FoxP3+ Tregs was higher in the LN (9.2%62.3% versus 5.1%62.1%; P=0.05) but was comparable in the thymus (2.6%60.9% versus 2.0%60.7%; P=0.42) of CD4-Keap1-KO mice compared with Keap1F/F mice (FIG. 3A). The frequency of all Foxp3+ cells was significantly higher in the LN of CD4-Keap1-KO (4.8%60.6% versus 2.6%61.4%; P=0.03) but was comparable in the thymus (FIG. 9A). Baseline intracellular IFN-g and TNF-a in CD8 T cells isolated from the LN and thymus of CD4-Keap1-KO mice were significantly attenuated (FIGS. 3, B and C, respectively). We did not observe any significant difference in the frequency of CD4, CD8, DNT, and double-positive populations in thymocytes between CD4-Keap1-KO and Keap1F/F mice, suggesting that T cell-specific augmentation of Nrf2 does not affect phenotypic diversity in T cell development (FIG. 9B).

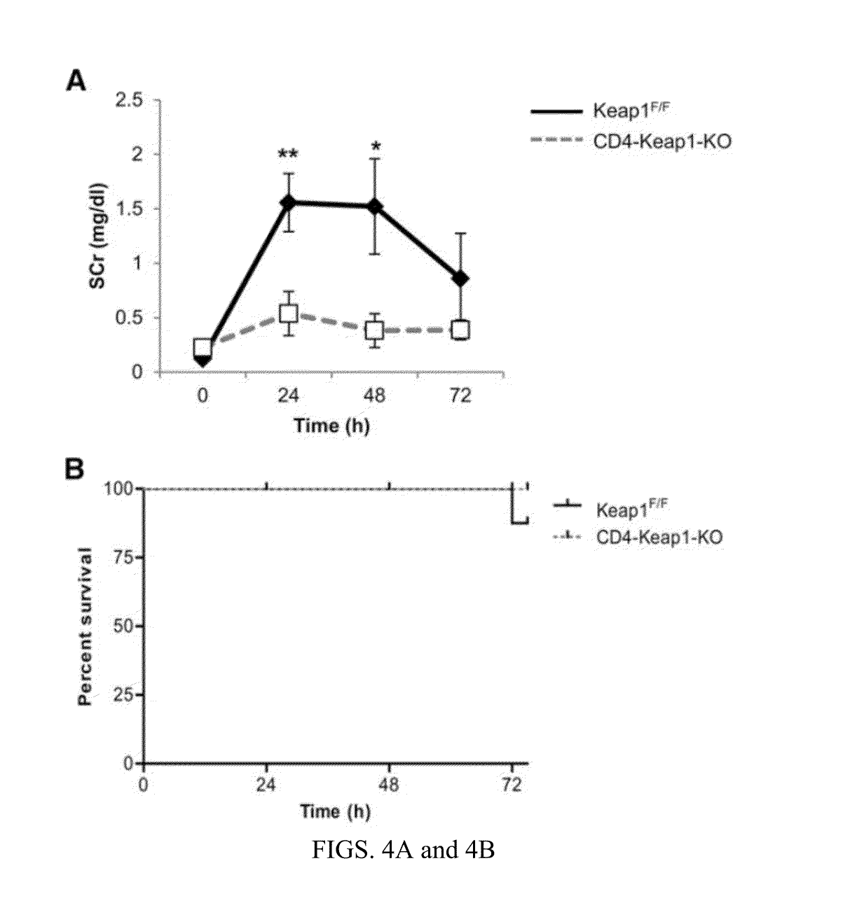

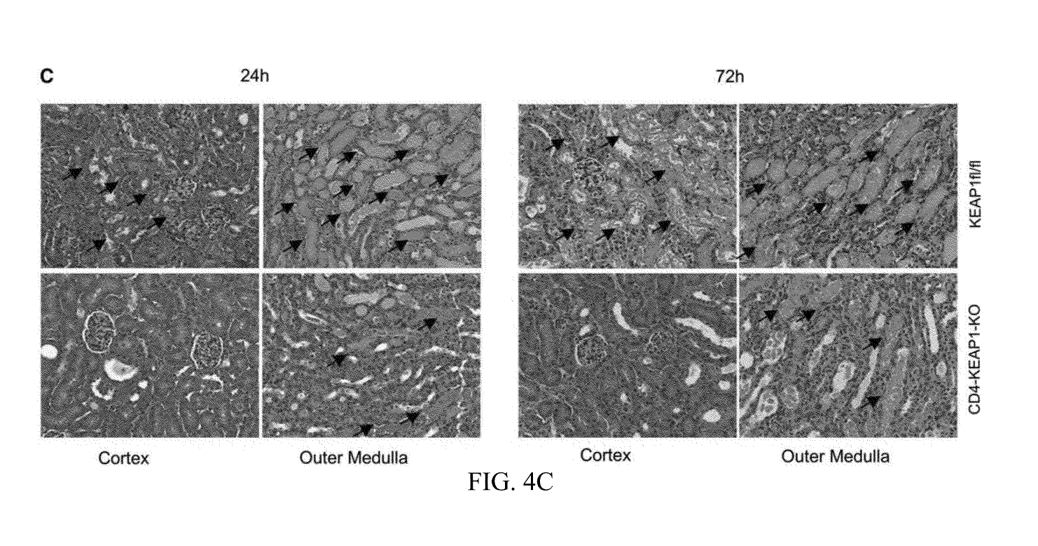

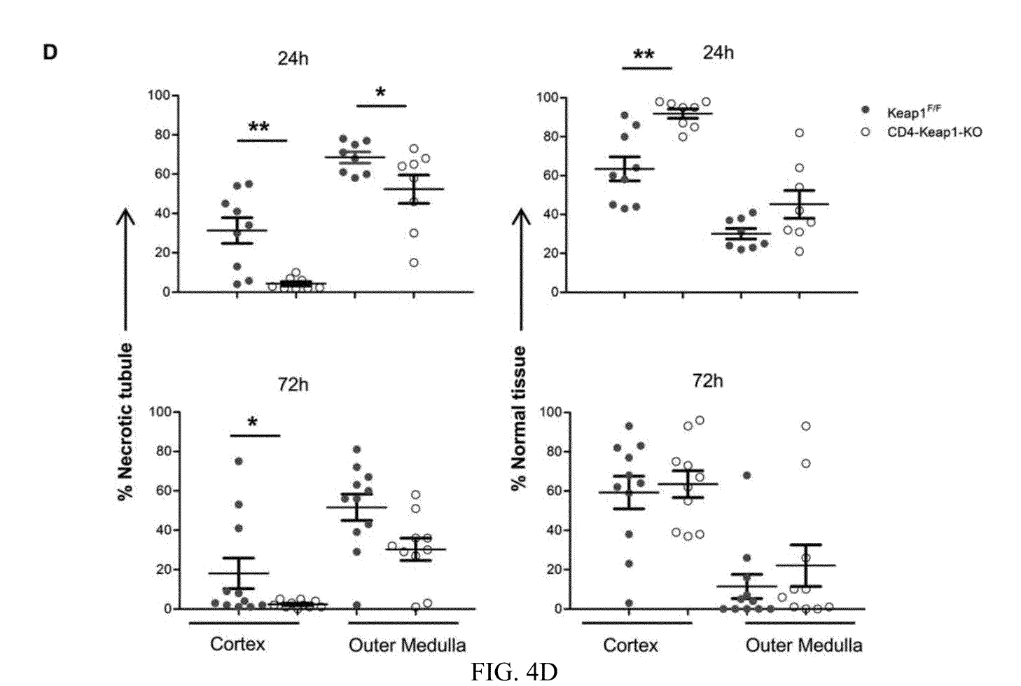

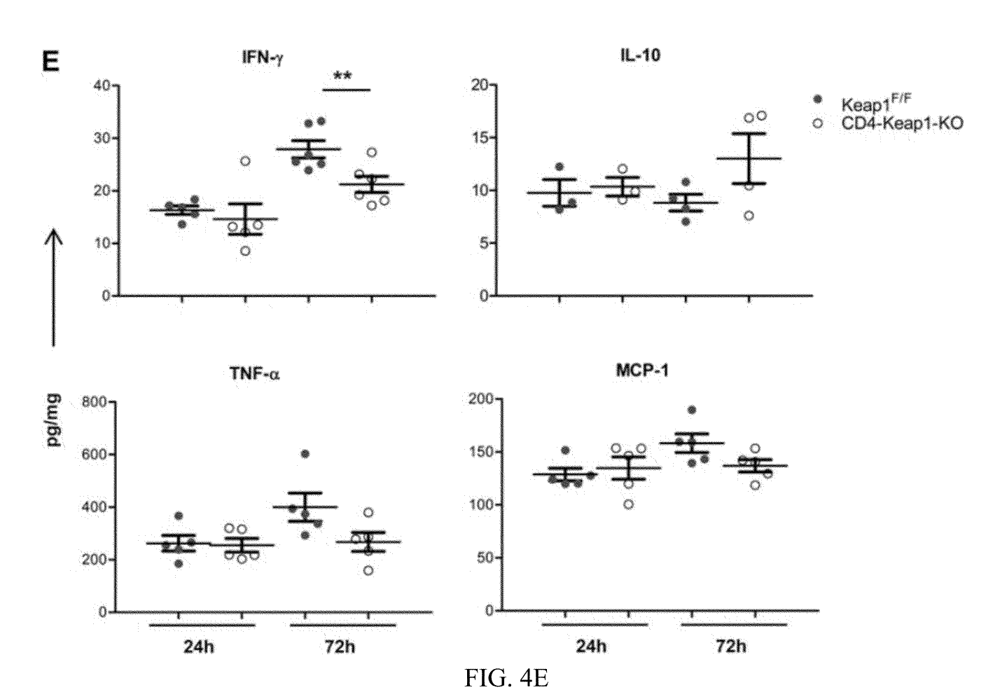

T Cell-Specific Augmentation of Nrf2 Protects Kidneys from IR Injury. To further investigate the effect of T cell-specific Nrf2 activation on IR-induced AKI, we subjected CD4-Keap1-KO and Keap1F/F mice to a well-established IRI model and evaluated structural and functional markers of kidney injury. We induced AKI by bilateral renal pedicle occlusion for 30 minutes followed by reperfusion. Increased antioxidant response in T cells in CD4-Keap1-KO mice resulted in significant protection from AKI compared with Keap1F/F mice. CD4-Keap1-KO mice exhibited significantly improved kidney function compared with Keap1F/F mice, indicated by reduced serum creatinine (SCr) levels at 24 hours (P≦0.01) and 48 hours (P≦0.05) after IR injury (FIG. 4A). Furthermore, we observed no mortality in CD4-Keap1-KO mice, whereas approximately 20% of Keap1F/F mice died 72 hours after IR injury (FIG. 4B).

Histologic evaluation of kidney tissue assessed by an expert pathologist (L.C.R.) blinded to the mouse groups revealed significantly fewer necrotic tubules and more normal appearing tubules in cortical and outer medullary regions in CD4-Keap1-KO kidneys compared with Keap1F/F control kidneys (FIGS. 4, C and D). Pro-inflammatory cytokine analysis in the whole kidney lysate showed reduced levels of IFN-g (21.261.8 versus 27.961.8; P=0.01). There was no significant difference in the other cytokines studied, including TNF-a (267.66 36 versus 400.1653.5; P=0.07), monocyte chemoattractant protein-1 (136.965.9 versus 158.368.9; P=0.07), and the anti-inflammatory cytokine IL-10 (1362.4 versus 8.861.1; P=0.1) (FIG. 4E).

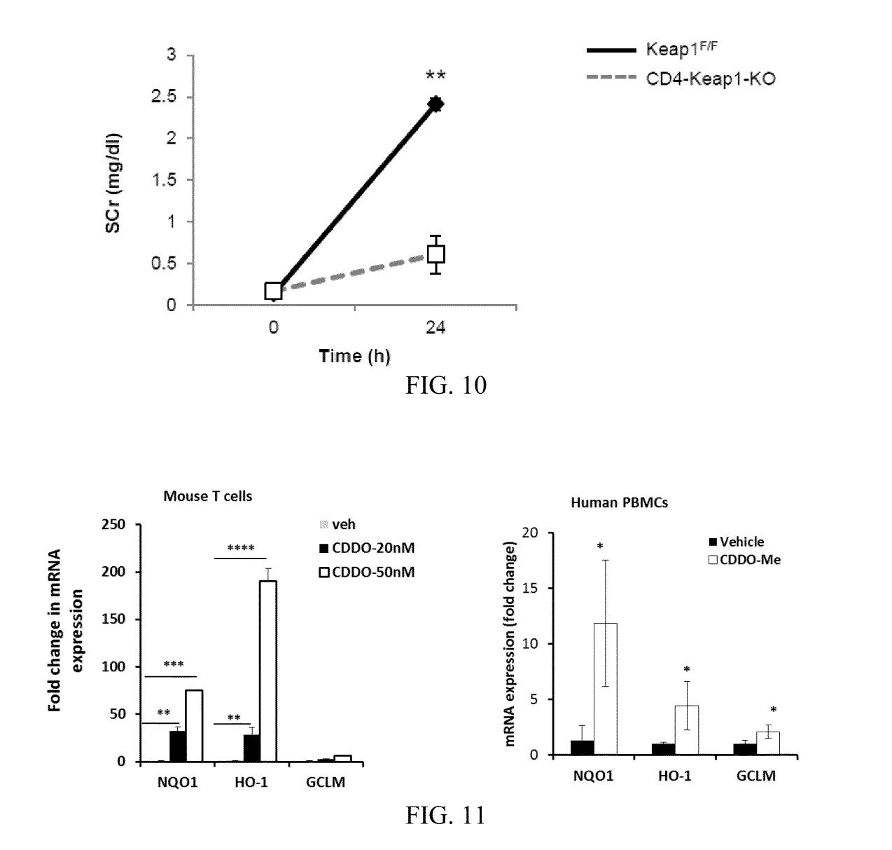

In an attempt to further understand the mechanism of structural and functional protection seen in CD4-Keap1-KO mice, we performed leukocyte phenotypic characterization and assessed intracellular cytokine levels in CD4-Keap1-KO and Keap1F/F mice after ischemic injury (FIG. 10). We observed a higher percentage of Tregs (6.1%62% versus 3.3%61.2%; P=0.04) and a lower percentage of CD11b+CD11c+(18.7%61.5% versus 23.6%61.8%; P=0.03) and F4/80+(34.9%61.8% versus 46.4%68.1%; P=0.03) cells in kidneys of CD4-Keap1-KO mice 24 hours after the induction of AKI (FIG. 5A). Absolute numbers of Tregs were not significantly different (343.306102.5 versus 284.1680.9 cells) in post-IR kidneys of CD4-Keap1-KO mice, nor were CD11b+CD11c+(6.4310461.83103 versus 8.3310463.23103 cells) and F4/80+(1310564.13103 versus 1.8310569.13103 cells) compared with Keap1F/F mice (FIG. 5B). Intracellular TNF-a and IFN-g were comparable in CD4, CD8 and DNT cells isolated from CD4-Keap1-KO and Keap1F/F kidneys; however, intracellular IL-17 production was significantly higher from CD4 (6.7%62.6% versus 2.9%60.9%; P=0.03) and DNT (8%62.7% versus 3.2%62%; P=0.03) cells isolated from CD4-Keap1-KO kidneys (FIG. 5C).

Augmentation of Nrf2 Decreases IFN-g but does not Affect IL-4 Production by CD4+ T Cells.

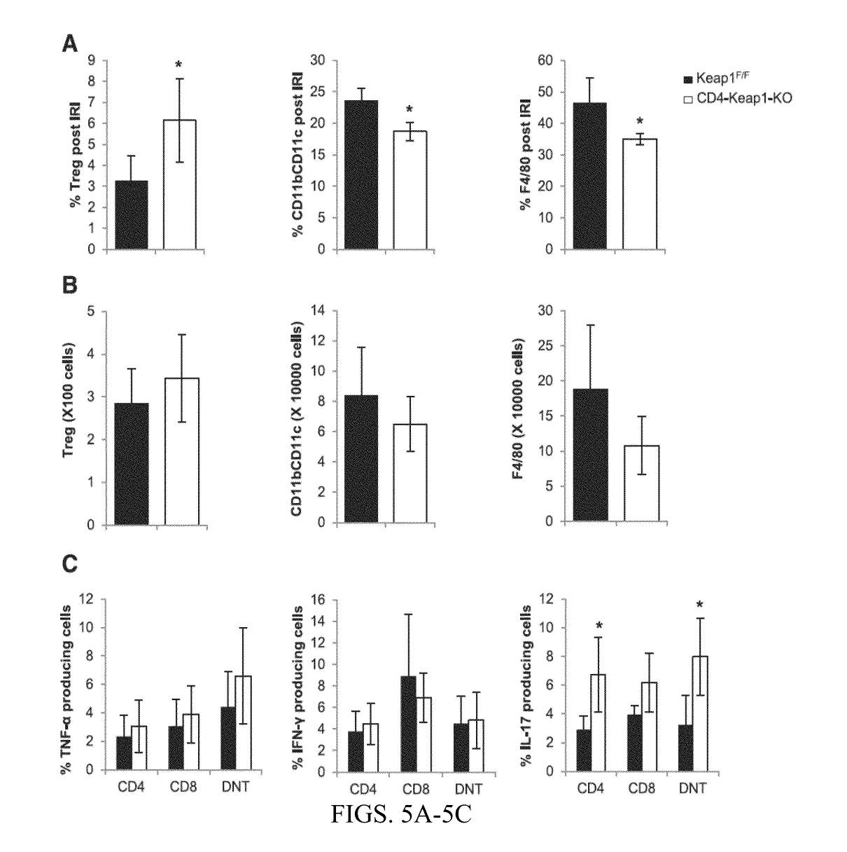

Based on the protection seen in our AKI model and in vivo intracellular data at baseline, we hypothesized that continuous Nrf2 activation in CD4-Keap1-KO mice resulted in T helper (Th) 2 type skewing in CD4+ T cells. Pharmacologic augmentation of Nrf2 has been shown to skew T cells toward the Th2 type that produces low levels of IFN-g and high levels of IL-4.24 To test our hypothesis that T cell-specificNrf2 activation by deleting Keap1 results in Th cell skewing, we purified CD4+ T cells from spleens of CD4-Keap1-KO and Keap1F/F mice and activated them in vitro with anti-CD3/CD28 antibodies under non-polarizing conditions (without anti-IFN-g and anti-IL-4) and measured intracellular levels of IFN-g and IL-4 by flow cytometry. Consistent with our in vivo data and previously published data, 24, 25 we observed significantly fewer IFNg-producing CD4+ T cells at day 3 (P=0.03) and day 7 (P=0.05) in CD4-Keap1-KO mice compared with Keap1F/F counterparts. However, there was no difference in the IL-4-producingCD4+Tcell population in either CD4-Keap1-KO or Keap1F/F mice (FIG. 6).

Adoptive Transfer of T Cells from CD4-Keap1-KO Mice Protects WT Mice from AKI and Improves Survival.

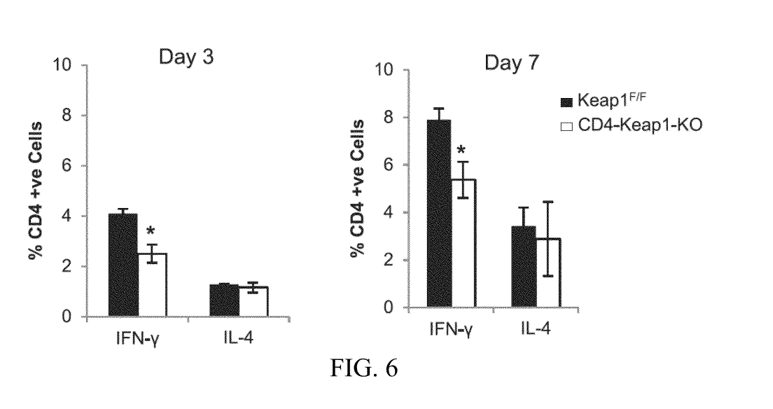

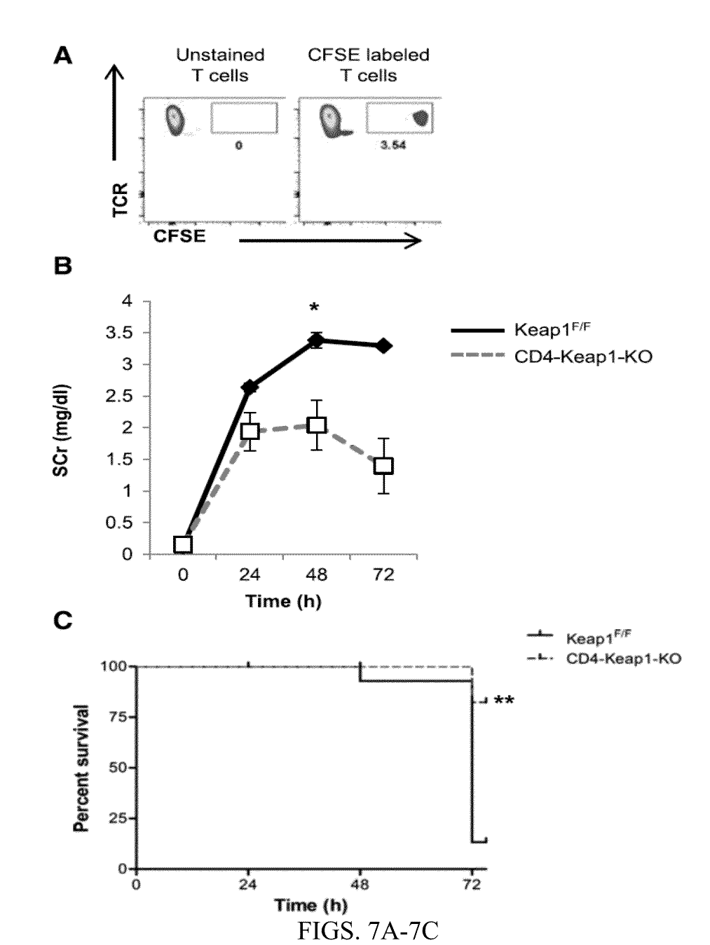

To further test the hypothesis that T cell-specific activation of Nrf2 pathway protects from IR injury and to explore its clinical therapeutic relevance, we transferred T cells from CD4-Keap1-KO and Keap1F/F mice into WT C57B1/6 mice by tail vein injection 24 hours before inducing AKI. The success of adoptive transfer was confirmed by establishing the presence of Carboxy fluorescein succinimidyl ester (CFSE) labeled T cells in peripheral blood of WT recipients before inducing AKI (FIG. 7A). We observed a significant (P≦0.02) improvement in kidney function in WT mice receiving T cells from CD4-Keap1-KO mice, as indicated by reduced SCr levels (FIG. 7B). Furthermore, adoptive transfer of T cells from CD4-Keap1-KO mice significantly (P≦0.01) improved survival of recipient WT mice after IR-induced AKI (FIG. 7C).

### Discussion

T lymphocytes play an important pathophysiologic role in modulating ischemic and nephrotoxic AKI. T cells are present in the kidney during both ischemia and reperfusion, and thus are significantly exposed to various oxidant species that can modulate their function. In this study, we generated mice with genetically upregulatedNrf2 in T cells and tested them in an IR model of AKI. Our data demonstrate that T cell-specific augmentation of Nrf2 increases antioxidant response and affects phenotypic diversity, activation, and recruitment of immune cells and reduces intracellular cytokine production by T cells in the kidneys. Importantly, Nrf2 activation in T cells provides significant protection against IR-induced AKI and improves survival. Furthermore, adoptive transfer of Nrf2-activated T cells to WT mice improves outcomes from AKI.

We observed many differences in CD4-Keap1-KO mice compared with Keap1F/F mice that may be responsible for the protection seen in our experimental AKI model. Frequency of Tregs was significantly higher in CD4-Keap1-KO mice, whereas CD11b+CD11c+DCs and F4/80+ macrophages were significantly reduced at baseline. Similar trends were observed for these cell types at 24 hours after IR injury, whereas Treg frequency remained significantly higher in CD4-Keap1-KO mice. These cell types are known to affect IR-induced kidney injury and repair. Multiple studies have demonstrated that Tregs promote post-ischemic kidney preconditioning and repair, suppress rejection, and induce allograft tolerance in kidney transplantation. Alternately, DCs and macrophages have been shown to worsen ischemic injury through various mechanisms and depletion of these cells protects the kidney from IR injury. Although numbers of DCs and macrophages increased after IR compared with baseline kidneys, their number was low in kidneys of CD4-Keap1-KOmice in this study. Attenuated DC and macrophage numbers in kidneys of CD4-Keap1-KO mice could be a direct effect of higher numbers of Tregs that regulate DCs and macrophage recruitment in the ischemic tissue. In addition, an increased Treg frequency in lymphoid tissue may provide a mechanism to attenuate inflammation during kidney IRI. Furthermore, T cells from CD4-Keap1-KO mice produced fewer pro-inflammatory cytokines than Keap1F/F mice. Although the exact mechanism by which T cell-specific Nrf2 activation ameliorates pro-inflammatory cytokine secretion is not clear, pharmacologic activation of Nrf2 with tertbutylhydroquinone and butylated hydroxyanisole was shown to skew T cells toward a Th2 type phenotype and suppress TNF-a and IFN-g production after CD3/CD28 activation. We did not observe any Th2 skewing per se; nonetheless, purified CD4+ T cells from CD4-Keap1-KO mice produced less IFN-g after in vitro CD3/CD28 activation, which is in concordance with our in vivo intracellular cytokine data at baseline and after IR injury. Li et al. recently demonstrated that activation of DCs with adenosine protects from AKI through modulation of natural killer (NK) T cell function and by attenuating IFN-g secretion, accompanied by increased IL-10 levels and subsequently reduced post-ischemic inflammation. Because we observed reduced IFN-g in post-IR kidneys of CD4-Keap1-KO mice, similar downstream effects along with phenotypic changes during AKI could be responsible for the protection from IR injury observed in this study. Furthermore, adoptive transfer experiments demonstrate that these T cells exert a strong protective effect given that they were transferred to WT mice with normal Nrf2 levels in T cells.

The pathogenesis of IR injury is complex and there is likely intricate crosstalk between multiple immune cells via production of cytokines, chemokines, oxygen free radicals, complement, and coagulant factors that accentuates tissue damage. Both NADPH oxidase and mitochondrial reactive oxygen species play critical pathophysiologic roles in AKI, but how T cell-specific Nrf2 augmentation affects these processes is not clear from this study. Recent studies demonstrate that engagement of TCR induces rapid production of reactive oxygen species and further modifies T cell signal transduction and gene expression. Oxidative stress further promotes T cell differentiation toward the Th2 phenotype under polarizing conditions. Additional data demonstrate that redox modulation suppresses CD8 T cell response to alloantigen and the TCR transgenic CD8 T cell responds to its cognate antigen by inhibiting proliferation, pro-inflammatory cytokine synthesis, and cytotoxic T lymphocyte effector mechanisms. In vitro-derived Th1 and Th2 clones or T cells derived from autoimmune thyroiditis have been shown to expand and produce cytokines in an oxidative environment. Furthermore, T cells are a heterogeneous group of cells with diverse functions and their interaction with DCs and macrophages during an ischemic event dictates the injury outcome. In this study, we observed attenuation of pro-inflammatory cytokines by CD4 and CD8 T cells, as well as by DNT cells. However, there are subtle differences in how different cytokines are regulated in the different lymphocytes by the Keap1 deletion. The response by DNT cells is particularly interesting because we did not predict them to be affected by CD4-Cre-mediated deletion of Keap1. This may be an effect of their interaction with other immune cells and overall cytokine milieu. Furthermore, a proportion of DNT cells may represent the reverting double-positive (CD4+CD8+) T cells. Therefore, T cell-specific activation of Nrf2-regulated antioxidant response appears to help in the maintenance of a low pro-inflammatory environment and optimal T cell function that subsequently results in reduced oxidative and inflammatory tissue injury. It is important to note that Nrf2-independent effects of Keap1 deletion in T lymphocytes may also be involved. Keap1 acts as an adapter protein for the E3 ubiquitin ligase complex that directs multiple proteins, including Nrf2, for proteasomal degradation. In addition, Keap1 has complex interactions with many other proteins that regulate NF-kB activation, T cell proliferation, integrin expression, and perforin production in NK cells. Keap1 has been linked to inflammation, autophagy, and apoptosis. Further experiments are warranted to identify Nrf2-independent effects of Keap1 deletion in T cells.

In summary, augmented antioxidant response in CD4-Keap1-KO mice increased the basal Treg population, reduced numbers of CD11b+CD11c+ and F4/80+ cells and promoted an anti-inflammatory environment in the kidney. These results demonstrate that basal Nrf2 levels in T cells have widespread effects on immune cell activation and injury outcome after an ischemic event. Despite the promise of Nrf2 targeting for kidney diseases, the search for an optimal Nrf2 activator is ongoing. Our data demonstrate that adoptively transferred T cells from CD4-Keap1-KO mice produced a strong protective effect in the WT mice; thus, activation of Nrf2 in T cells holds promise for immune cell therapy.

Development of Novel Keap1 Deletion Strategies for T Lymphocyte Based Therapies in AKI.

T lymphocytes respond to antigens presented by bona fide antigen presenting cells and are generally not considered to respond to other stimuli such as oxidative stress. However, recent data, including ours, is indicating that T lymphocyte may be important not only for reactive oxygen production but also the way they scavenge these ROS. We found that activating Nrf2 activity by deleting its inhibitor Keap1 leads to significant protection from IR induced AKI. Designing novel Nrf2 activation or keap1 deletion strategies is very important for successful targeting of T lymphocyte specific Nrf2 pathway in AKI and kidney disease. In the following experiments we will make an attempt to investigate additional Nrf2 activation/keap1 deletion methods both in mouse and human T cells.

Experimental Design:

We will isolate pure T cells from mouse spleen using T cell specific antibodies on magnetic beads. In one approach, we will treat purified T cells with Nrf2 activator molecules such as Sulforaphane, tBHq, Protandim, Chalcone derivatives, triterpenoid derivatives such as CDDO-Im and CDDO-Me. Although we found that CDDO-Im at 20 and 50 μM to be significantly effective in activating Nrf2 regulated antioxidant gene expression in purified mouse T cells and human PBMCs, we will try multiple doses and times to activate T cell-Nrf2 with different pharmacologic activators in this study in order to arrive at the most suitable dose and time point for Nrf2 activation. In second approach we will use Cas9 ribonucleoproteins (Cas9 RNPs) specifically designed against mouse Keap1 gene. Briefly, we will design Keap1 specific RNPs and assemble them with Cas9 protein immediately before transferring them in to T cells using electroporation. Electroporated T cells will be transferred to CD3/CD28 coated plates for 2 h at 37° C. and subsequently resuspended and transferred to a non-coated plate. T cell s will be analyzed for T7 endonuclease activity 3-4 days after electroporation to confirm the gene deletion. T cells with stable Keap1 deletion will be enriched using FACS and analyzed for Nrf2 regulated antioxidant response by assessing mRNA levels of Nrf2 target genes (NQO1, HO1, GCLC and GCLM) and expanded for additional experiments discussed herein. In addition to Cas9 RNPs, we will also employ Keap1 specific siRNAs to delete Keap1 in primary T cells from mouse. Keap1 specific siRNA against mouse Keap1 gene is commercially available (Santa Cruz) and will be used for these studies. Briefly, pure T cells isolated using magnetic beads will be treated with Keap1 specific siRNA for 5-7 hours at 37° C. in a CO₂ incubator, washed and incubated in normal growth medium for additional 18-24 hours and assayed for Nrf2 regulated antioxidant gene expression using real-time PCR. Finally we will treat primary mouse T cells using miRNAs against Keap1 RNA (miRNA 200a). Briefly, Keap1 specific miRNA (miRNA-200a) will be transfected with Lipofectamine 2000 according to the manufacturer's instruction. The nucleotides of miRNA-200a mimic and miRNA-200a inhibitor will be used at a concentration of 60 nM and 80 nM respectively in antibiotic-free Opti-MEM medium. After 6 h, the medium will be replaced with DMEM with 10% FBS, without antibiotics. RNA will be extracted after 48 h of transfection and antioxidant gene expression assessed using real-time PCR.

It is expected that Nrf2 activation using various pharmacologic activators and novel Keap1 deletion methods will results in a robust up-regulation of Nrf2 regulated antioxidant genes in primary mouse T cells.

To Investigate the Effect of Different Keap1 Deletion Strategies on AKI Outcome in IR and Cisplatin-Mouse Model of AKI.

We will test whether transfer of T cells following ex-vivo Nrf2 activation/Keap1 deletion protects from IR and cisplatin induced AKI. We will use 6-8 wk old WT mice (n=10 per group) for these experiment. Approximately 10×10⁶ ex vivo treated T cells will be transferred to WT mice, 24 h before inducing AKI. Briefly, AKI will be induced by well-established 30 minute bilateral IR surgery or cisplatin injection (30 mg/kg). We will assess kidney function by measuring serum creatinine (SCr) at 0 h (baseline), 24 h, 48 h and 72 h post IRI surgery or cisplatin injection to determine the protective effect of novel pharmacologic Nrf2 activation/Keap1 deletion strategy. Control mice will receive either PBS or untreated T cells and subjected to IR or cisplatin induced AKI similar to those described for experimental groups. At 72 h mice will be euthanized and kidney will be evaluate for histological damage using H&E staining, infiltration of inflammatory cells and cytokines by flow cytometry, Kidney ROS (SOD, Phospho-IkB/NF-kB) and Nrf2 target gene expression (HO-1, NQO1, GCLC/GCLM) to assess the effect of different Nrf2 activation/Keap1 deletion strategies on AKI outcome.

It is expected that transfer of T cells that underwent ex vivo Nrf2 activation or Keap1 deletion will protect kidneys from IR and cisplatin induced AKI. We expect the mice in the experimental groups will show a better kidney function, less inflammation, increased antioxidant response and reduced histological damage.

Examine the Effect of Nrf2 Activation/Keap1 Deletion Strategy in Human Primary T Lymphocytes.

We will test whether Nrf2 activation/Keap1 deletion strategies described herein and found most effective in abrogating AKI can be used to activate Nrf2 in human primary T lymphocytes. In an approach similar to that described herein, we will isolate pure T cells from human peripheral blood using magnetic beads. We will treat purified T cells with Nrf2 activator molecules, human T cells specific Cas9 ribonucleoproteins (Cas9 RNPs) against Keap1 gene (Cas9 RNPs have recently used to successfully knock out and knock in genes in primary human T cells), Keap1 specific siRNAs to delete Keap1 in primary T cells from human (Keap1 specific siRNA against human Keap1 gene are commercially available and will be used for these studies) Keap1 specific miRNA against human keap1 gene. We will then examine the effect of these Nrf2 activation/deletion strategies on Nrf2 regulated antioxidant response using real-time PCR, phenotypic and functional characterization of T cell and their ability to bear oxidative stress using in vitro methods.

It is expected that Nrf2 activation using various pharmacologic activators and novel Keap1 deletion methods will result in a robust up-regulation of Nrf2 regulated antioxidant genes and may result in a TH2 type phenotype or increased expression of FoxP3 as well as reduction in pro-inflammatory cytokine production. We further expect that these cells will better handle an exogenous oxidative insult (H₂O₂ etc.) than untreated T cells.

We claim:

1. A knockout animal whose genome comprises a deletion of exon 2 and exon 3 of kelch-like ECH-associated protein 1 (KEAP1) in T-cells.

2. The knockout animal of claim 1, wherein KEAP1 is encoded by the nucleic acid sequence of SEQ ID NO:9.

3. The knockout animal of claim 1, wherein exon 2 is encoded by the nucleic acid sequence of SEQ ID NO:10.

4. The knockout animal of claim 1, wherein exon 3 is encoded by the nucleic acid sequence of SEQ ID NO:11.

5. The knockout animal of claim 1, wherein the animal exhibits lower or no expression of KEAP1 as compared to a wildtype animal.

6. The knockout animal of claim 1, wherein the animal is a mouse.

7. The knockout animal of claim 1, wherein the animal is a rat.

8. A population of T-cells derived or isolated from the knockout animal of claim 1.

9. A method comprising the steps of:
a. activating Nrf2 in T-cells isolated from a subject; and
b. administering the T-cells of step (a) to the subject.

10. The method of claim 9, wherein the subject is a human.

11. The method of claim 9, wherein the subject suffers from acute kidney injury (AKI).

12. The method of claim 11, wherein the subject suffers from ischemia reperfusion induced AKI.

13. The method of claim 9, wherein the activating step is accomplished by contacting the T-cells with an Nrf2-activator.

14. The method of claim 13, wherein the Nrf2 activator is sulforaphane.

15. The method of claim 13, wherein the Nrf2 activator is one or more of tert-butylhydroquinone (tBHQ), Protandim, Cddo-Im, CDDO-Me, Oltipraz (4-methyl-5-(2-pyrazinyl)-3-dithiolethione), bardoxolone methyl, dihydro-CDDO-trifluoroethyl amide (dh404), resveratrol, chalcone, a chalcone derivative, anethole dithiolethione, 6-methylsulphinylhexyl isothiocyanate, curcumin, caffeic acid phenethyl ester, and 4′-bromoflavone.

16. A method for treating a subject diagnosed with AKI comprising the steps of:
a. isolating T-cells from the subject;
b. activating Nrf2 expression in the isolated T-cells; and
c. administering the T-cells back to the subject.

17. The method of claim 16, wherein the AKI comprises ischemia reperfusion induced AKI.

18. The method of claim 16, wherein the activating step is accomplished by contacting the T-cells with an Nrf2-activator.

19. The method of claim 18, wherein the Nrf2 activator is sulforaphane.

20. The method of claim 18, wherein the Nrf2 activator is one or more of tert-butylhydroquinone (tBHQ), Protandim, Cddo-Im, CDDO-Me, Oltipraz (4-methyl-5-(2-pyrazinyl)-3-dithiolethione), bardoxolone methyl, dihydro-CDDO-trifluoroethyl amide (dh404), resveratrol, chalcone, a chalcone derivative, anethole dithiolethione, 6-methylsulphinylhexyl isothiocyanate, curcumin, caffeic acid phenethyl ester, and 4′-bromoflavone.

21. A method for treating a subject diagnosed with AKI comprising the step of administering to the subject autologous T-cells that were previously isolated from the subject and treated ex-vivo to activate Nrf2 expression.

22. The method of claim 21, wherein the treatment step is accomplished by contacting the T-cells with an Nrf2-activator.

23. The method of claim 22, wherein the Nrf2 activator is sulforaphane.

24. The method of claim 22, wherein the Nrf2 activator is one or more of tert-butylhydroquinone (tBHQ), Protandim, Cddo-Im, CDDO-Me, Oltipraz (4-methyl-5-(2-pyrazinyl)-3-dithiolethione), bardoxolone methyl, dihydro-CDDO-trifluoroethyl amide (dh404), resveratrol, chalcone, a chalcone derivative, anethole dithiolethione, 6-methylsulphinylhexyl isothiocyanate, curcumin, caffeic acid phenethyl ester, and 4′-bromoflavone.

25. A method for treating a patient having an ischemia-related injury comprising the steps of administering the subject autologous T-cells that were previously isolated from the subject and treated ex-vivo to activate/upregulate Nrf2 expression.

26. The method of claim 25, wherein the ischemia-related injury comprises organ injuries suffered during transplantation, myocardial infraction, hemorrhage, cardiac arrest and other oxidative stress and inflammation driven diseases.

27. The method of claim 25, wherein the treatment step is accomplished by contacting the T-cells with an Nrf2-activator.

28. The method of claim 27, wherein the Nrf2 activator is sulforaphane.

29. The method of claim 27, wherein the Nrf2 activator is one or more of tert-butylhydroquinone (tBHQ), Protandim, Cddo-Im, CDDO-Me, Oltipraz (4-methyl-5-(2-pyrazinyl)-3-dithiolethione), bardoxolone methyl, dihydro-CDDO-trifluoroethyl amide (dh404), resveratrol, chalcone, a chalcone derivative, anethole dithiolethione, 6-methylsulphinylhexyl isothiocyanate, curcumin, caffeic acid phenethyl ester, and 4′-bromoflavone.
